(19) United States
(12) Patent Application Publication
(10) Pub. No.: US 2014/0259232 A1
(43) Pub. Date: Sep. 11, 2014

(54) MOLECULAR MARKERS ASSOCIATED WITH EARLINESS IN MAIZE

(71) Applicant: Dow AgroSciences LLC, Indianapolis, IN (US)

(72) Inventors: Rajesh Perianayagam, Indianapolis, IN (US); Jan E. Backlund, Indianapolis, IN (US); Stipe Vujevic, St. Marys (CA); Trisha Borowicz, Greenfield, IN (US); Chibwe Chungu, Willmar, MN (US); David H. Meyer, Indianapolis, IN (US); Bruce A. Nagel, Beaver Dam, WI (US); Othmar M. Frei, Bruck (AT); John P. Davies, Portland, OR (US)

(73) Assignee: DOW AGROSCIENCES LLC, Indianapolis, IN (US)

(21) Appl. No.: 14/197,249

(22) Filed: Mar. 5, 2014

Related U.S. Application Data
(60) Provisional application No. 61/774,620, filed on Mar. 8, 2013.

(51) Int. Cl. C12Q 1/68

(52) U.S. Cl. CPC C12Q 1/6895
USPC 800/320.1; 435/6.11

(57) ABSTRACT

This invention relates to methods for identifying maize plants that having decreased flowering time. The methods use molecular markers to identify and to select plants with decreased flowering time. Maize plants generated by the methods of the invention are also a feature of the invention.

This application claims a priority based on provisional application 61/774,620 which was filed in the U.S. Patent and Trademark Office on Mar. 8, 2013, the entire disclosure of which is hereby incorporated by reference.

### FIELD OF THE INVENTION

The present invention relates to methods useful in selecting forearliness in maize plants.

### BACKGROUND OF THE INVENTION

Due to increasing food production needs and heightened demands for plant biomass as a source of renewable energy created by a burgeoning global population, a significant amount of biotechnology research is being devoted to increasing the yield of corn plants. Timing of flowering can have a significant impact on production of agricultural products. For example, corn varieties with different flowering times are necessary to adapt crops to different production regions or systems. Flowering time is an agronomically important trait that is linked to the adaptation to different environmental conditions, tolerance to biotic, abiotic stresses and yield in corn. Genes that control flowering time affect hybrid vigor and thus are likely to impact on yield. To meet the increased demands of the growing market, corn acres have expanded into areas of the northern great plains of the United States, previously planted to more cool season cereal crops such as wheat and barley. Corn grown in these “non-traditional” areas of northwestern Minnesota and North Dakota face a much cooler and shorter growing season than corn grown across the traditional US Corn Belt. Therefore, the need arises to develop early flowering corn hybrids and parent lines that will mature in the cooler and shorter growing season of this region. The present invention addresses this need and provides improved methods for decreasing flowering time will facilitate the development of new, geographically adapted corn varieties.

### SUMMARY OF THE INVENTION

One embodiment of the present invention is a method for selecting a plant having an altered flowering characteristic. The method includes the steps of: a) detecting at least one marker nucleic acid; and, b) selecting a plant comprising the marker nucleic acid, thereby selecting a plant having the altered flowering characteristic. The plant is preferably a maize plant. The altered flowering characteristic is altered flowering time, preferably decreased flowering time. In embodiments of the invention, the flowering time is decreased by at least 2 days.

In embodiments of the invention, the marker nucleic acid is selected from the group consisting of DAS-PZ-10450, DAS-PZ-12076, DAS-PZ-16344, DAS-PZ-1867, DAS-PZ-2256, DAS-PZ-6992, DAS-PZ-8291, DAS-PZ-9937, DSDS-0066-1, KG-2679163, magi18365, Mo17-101121, PZA03746.1, PZA02737.1, and DAS-PZ-6563. In other embodiments of the invention, the marker nucleic acid is selected from the group consisting of DAS-PZ-10083, DAS-PZ-1255, DAS-PZ-14161, DAS-PZ-15602, DAS-PZ-19469, DAS-PZ-3132, DAS-PZ-4347, DAS-PZ-5837, DAS-PZ-8111, DAS-PZ-880, DAS-PZ-9899, magi99055, Mo17-10988, Mo17-11370, Mo17-11923, Mo17-13135, Mo17-1316, Mo17-13288, PHM13823.7, PHM2324.23, PZA03070.9, and PZA00279.2. In further embodiments, the marker nucleic acid is selected from the group consisting of DAS-PZ-2559, DAS-PZ-3168, DAS-PZ-3394, magi104662, magi52178, Mo17-10465, PHM6428.11, PZA01470.1 and DAS-PZ-9978. In other embodiments, the marker nucleic acid is selected from the group consisting of DAS-PZ-366, KG-2566510, KG-2624948, Mo17-13418, PZA00466.1, PZA01272.1, PZB01899.1, and PZB01963.1.

In embodiments of the invention, at least two marker nucleic acids are selected, preferably, at least three marker nucleic acids are selected, more preferably at least four marker nucleic acids are selected.

In yet another embodiment of the invention is a method for selecting a maize plant having decreased flowering time, the method comprising:

a) detecting at least four marker nucleic acids, wherein at least one marker nucleic acid is selected from each of four marker nucleic acid groups (i)-(iv): (i) DAS-PZ-10450, DAS-PZ-12076, DAS-PZ-16344, DAS-PZ-1867, DAS-PZ-2256, DAS-PZ-6992, DAS-PZ-8291, DAS-PZ-9937, DSDS-0066-1, KG-2679163, magi18365, Mo17-101121, PZA03746.1, PZA02737.1, and DAS-PZ-6563;
(ii) DAS-PZ-10083, DAS-PZ-1255, DAS-PZ-14161, DAS-PZ-15602, DAS-PZ-19469, DAS-PZ-3132, DAS-PZ-4347, DAS-PZ-5837, DAS-PZ-8111, DAS-PZ-880, DAS-PZ-9899, magi99055, Mo17-10988, Mo17-11370, Mo17-11923, Mo17-13135, Mo17-1316, Mo17-13288, PHM13823.7, PHM2324.23, PZA03070.9, and PZA00279.2;
(iii) DAS-PZ-2559, DAS-PZ-3168, DAS-PZ-3394, magi104662, magi52178, Mo17-10465, PHM6428.11, PZA01470.1, and DAS-PZ-9978; and,
(iv) DAS-PZ-366, KG-2566510, KG-2624948, Mo17-13418, PZA00466.1, PZA01272.1, PZB01899.1, and PZB01963.1; and,

b) selecting a plant comprising the four marker nucleic acids, thereby selecting a maize plant having decreased flowering time. Maize plants obtained by the methods described herein are also contemplated by the present invention.

### BRIEF DESCRIPTION OF FIGURES AND SEQUENCE LISTINGS

The invention can be more fully understood from the following detailed description and the accompanying drawings and Sequence Listing, which form a part of this application. The Sequence Listing contains the one letter code for nucleotide sequence characters and the three letter codes for amino acids as defined in conformity with the IUPAC-IUBMB standards described in Nucleic Acids Research 13:3021-3030 (1985) and in the Biochemical Journal 219 (No. 2): 345-373 (1984), which are herein incorporated by reference in their entirety. The symbols and format used for nucleotide and amino acid sequence data comply with the rules set forth in 37 C.F.R. §1.822.

SEQ ID NO: 1 contains the DAS-PZ-10450 SNP and flanking sequence.

SEQ ID NO: 2 contains the DAS-PZ-12076 SNP and flanking sequence.

SEQ ID NO: 3 contains the DAS-PZ-16344 SNP and flanking sequence.

SEQ ID NO: 4 contains the DAS-PZ-1867 SNP and flanking sequence.

SEQ ID NO: 5 contains the DAS-PZ-2256 SNP and flanking sequence.

SEQ ID NO: 6 contains the DAS-PZ-6992 SNP and flanking sequence.

SEQ ID NO: 7 contains the DAS-PZ-8291 SNP and flanking sequence.

SEQ ID NO: 8 contains the DAS-PZ-9937 SNP and flanking sequence.

SEQ ID NO: 9 contains the DSDS-0066-1 SNP and flanking sequence.

SEQ ID NO: 10 contains the KG-2679163 SNP and flanking sequence.

SEQ ID NO: 11 contains the Mo17-101121 SNP and flanking sequence.

SEQ ID NO: 12 contains the DAS-PZ-6563 SNP and flanking sequence.

SEQ ID NO: 13 contains the DAS-PZ-10083 SNP and flanking sequence.

SEQ ID NO: 14 contains the DAS-PZ-1255 SNP and flanking sequence.

SEQ ID NO: 15 contains the DAS-PZ-14161 SNP and flanking sequence.

SEQ ID NO: 16 contains the DAS-PZ-15602 SNP and flanking sequence.

SEQ ID NO: 17 contains the DAS-PZ-19469 SNP and flanking sequence.

SEQ ID NO: 18 contains the DAS-PZ-3132 SNP and flanking sequence.

SEQ ID NO: 19 contains the DAS-PZ-4347 SNP and flanking sequence.

SEQ ID NO: 20 contains the DAS-PZ-5837 SNP and flanking sequence.

SEQ ID NO: 21 contains the DAS-PZ-8111 SNP and flanking sequence.

SEQ ID NO: 22 contains the DAS-PZ-880 SNP and flanking sequence.

SEQ ID NO: 23 contains the DAS-PZ-9899 SNP and flanking sequence.

SEQ ID NO: 24 contains the Mo17-10988 SNP and flanking sequence.

SEQ ID NO: 25 contains the Mo17-11370 SNP and flanking sequence.

SEQ ID NO: 26 contains the Mo17-11923 SNP and flanking sequence.

SEQ ID NO: 27 contains the Mo17-13135 SNP and flanking sequence.

SEQ ID NO: 28 contains the Mo17-1316 SNP and flanking sequence.

SEQ ID NO: 29 contains the Mo17-13288 SNP and flanking sequence.

SEQ ID NO: 30 contains the DAS-PZ-2559 SNP and flanking sequence.

SEQ ID NO: 31 contains the DAS-PZ-3168 SNP and flanking sequence.

SEQ ID NO: 32 contains the DAS-PZ-3394 SNP and flanking sequence.

SEQ ID NO: 33 contains the magi104662 SNP and flanking sequence.

SEQ ID NO: 34 contains the Mo17-10465 SNP and flanking sequence.

SEQ ID NO: 35 contains the DAS-PZ-9978 SNP and flanking sequence.

SEQ ID NO: 36 contains the DAS-PZ-366 SNP and flanking sequence.

SEQ ID NO: 37 contains the KG-2566510 SNP and flanking sequence.

SEQ ID NO: 38 contains the KG-2624948 SNP and flanking sequence.

SEQ ID NO: 39 contains the Mo17-13418 SNP and flanking sequence.

SEQ ID NO: 40 is a forward PCR primer for the amplification of SEQ ID NO: 1.

SEQ ID NO: 41 is a forward PCR primer for the amplification of SEQ ID NO: 1.

SEQ ID NO: 42 is a reverse PCR primer for the amplification of SEQ ID NO: 1.

SEQ ID NO: 43 is a forward PCR primer for the amplification of SEQ ID NO: 2.

SEQ ID NO: 44 is a forward PCR primer for the amplification of SEQ ID NO: 2.

SEQ ID NO: 45 is a reverse PCR primer for the amplification of SEQ ID NO: 2.

SEQ ID NO: 46 is a forward PCR primer for the amplification of SEQ ID NO: 3.

SEQ ID NO: 47 is a forward PCR primer for the amplification of SEQ ID NO: 3.

SEQ ID NO: 48 is a reverse PCR primer for the amplification of SEQ ID NO: 3.

SEQ ID NO: 49 is a forward PCR primer for the amplification of SEQ ID NO: 4.

SEQ ID NO: 50 is a forward PCR primer for the amplification of SEQ ID NO: 4.

SEQ ID NO: 51 is a reverse PCR primer for the amplification of SEQ ID NO: 4.

SEQ ID NO: 52 is a forward PCR primer for the amplification of SEQ ID NO: 5.

SEQ ID NO: 53 is a forward PCR primer for the amplification of SEQ ID NO: 5.

SEQ ID NO: 54 is a reverse PCR primer for the amplification of SEQ ID NO: 5.

SEQ ID NO: 55 is a forward PCR primer for the amplification of SEQ ID NO: 6.

SEQ ID NO: 56 is a forward PCR primer for the amplification of SEQ ID NO: 6.

SEQ ID NO: 57 is a reverse PCR primer for the amplification of SEQ ID NO: 6.

SEQ ID NO: 58 is a forward PCR primer for the amplification of SEQ ID NO: 7.

SEQ ID NO: 59 is a forward PCR primer for the amplification of SEQ ID NO: 7.

SEQ ID NO: 60 is a reverse PCR primer for the amplification of SEQ ID NO: 7.

SEQ ID NO: 61 is a forward PCR primer for the amplification of SEQ ID NO: 8.

SEQ ID NO: 62 is a forward PCR primer for the amplification of SEQ ID NO: 8.

SEQ ID NO: 63 is a reverse PCR primer for the amplification of SEQ ID NO: 8.

SEQ ID NO: 64 is a forward PCR primer for the amplification of SEQ ID NO: 9.

SEQ ID NO: 65 is a forward PCR primer for the amplification of SEQ ID NO: 9.

SEQ ID NO: 66 is a reverse PCR primer for the amplification of SEQ ID NO: 9.

SEQ ID NO: 67 is a forward PCR primer for the amplification of SEQ ID NO: 10.

SEQ ID NO: 68 is a forward PCR primer for the amplification of SEQ ID NO: 10.

SEQ ID NO: 69 is a reverse PCR primer for the amplification of SEQ ID NO: 10.

SEQ ID NO: 70 is a forward PCR primer for the amplification of magi18365.

SEQ ID NO: 71 is a forward PCR primer for the amplification of magi18365.

SEQ ID NO: 72 is a reverse PCR primer for the amplification of magi18365.

SEQ ID NO: 73 is a forward PCR primer for the amplification of SEQ ID NO: 11.

SEQ ID NO: 74 is a forward PCR primer for the amplification of SEQ ID NO: 11.

SEQ ID NO: 75 is a reverse PCR primer for the amplification of SEQ ID NO: 11.

SEQ ID NO: 76 is a forward PCR primer for the amplification of PZA03746.1.

SEQ ID NO: 77 is a forward PCR primer for the amplification of PZA03746.1.

SEQ ID NO: 78 is a reverse PCR primer for the amplification of PZA03746.1.

SEQ ID NO: 79 is a forward PCR primer for the amplification of PZA02737.1.

SEQ ID NO: 80 is a forward PCR primer for the amplification of PZA02737.1.

SEQ ID NO: 81 is a reverse PCR primer for the amplification of PZA02737.1.

SEQ ID NO: 82 is a forward PCR primer for the amplification of SEQ ID NO: 12.

SEQ ID NO: 83 is a forward PCR primer for the amplification of SEQ ID NO: 12.

SEQ ID NO: 84 is a reverse PCR primer for the amplification of SEQ ID NO: 12.

SEQ ID NO: 85 is a forward PCR primer for the amplification of SEQ ID NO: 13.

SEQ ID NO: 86 is a forward PCR primer for the amplification of SEQ ID NO: 13.

SEQ ID NO: 87 is a reverse PCR primer for the amplification of SEQ ID NO: 13.

SEQ ID NO: 88 is a forward PCR primer for the amplification of SEQ ID NO: 14.

SEQ ID NO: 89 is a forward PCR primer for the amplification of SEQ ID NO: 14.

SEQ ID NO: 90 is a reverse PCR primer for the amplification of SEQ ID NO: 14.

SEQ ID NO: 91 is a forward PCR primer for the amplification of SEQ ID NO: 15.

SEQ ID NO: 92 is a forward PCR primer for the amplification of SEQ ID NO: 15.

SEQ ID NO: 93 is a reverse PCR primer for the amplification of SEQ ID NO: 15.

SEQ ID NO: 94 is a forward PCR primer for the amplification of SEQ ID NO: 16.

SEQ ID NO: 95 is a forward PCR primer for the amplification of SEQ ID NO: 16.

SEQ ID NO: 96 is a reverse PCR primer for the amplification of SEQ ID NO: 16.

SEQ ID NO: 97 is a forward PCR primer for the amplification of SEQ ID NO: 17.

SEQ ID NO: 98 is a forward PCR primer for the amplification of SEQ ID NO: 17.

SEQ ID NO: 99 is a reverse PCR primer for the amplification of SEQ ID NO: 17.

SEQ ID NO: 100 is a forward PCR primer for the amplification of SEQ ID NO: 18.

SEQ ID NO: 101 is a forward PCR primer for the amplification of SEQ ID NO: 18.

SEQ ID NO: 102 is a reverse PCR primer for the amplification of SEQ ID NO: 18.

SEQ ID NO: 103 is a forward PCR primer for the amplification of SEQ ID NO: 19.

SEQ ID NO: 104 is a forward PCR primer for the amplification of SEQ ID NO: 19.

SEQ ID NO: 105 is a reverse PCR primer for the amplification of SEQ ID NO: 19.

SEQ ID NO: 106 is a forward PCR primer for the amplification of SEQ ID NO: 20.

SEQ ID NO: 107 is a forward PCR primer for the amplification of SEQ ID NO: 20.

SEQ ID NO: 108 is a reverse PCR primer for the amplification of SEQ ID NO: 20.

SEQ ID NO: 109 is a forward PCR primer for the amplification of SEQ ID NO: 21.

SEQ ID NO: 110 is a forward PCR primer for the amplification of SEQ ID NO: 21.

SEQ ID NO: 111 is a reverse PCR primer for the amplification of SEQ ID NO: 21.

SEQ ID NO: 112 is a forward PCR primer for the amplification of SEQ ID NO: 22.

SEQ ID NO: 113 is a forward PCR primer for the amplification of SEQ ID NO: 22.

SEQ ID NO: 114 is a reverse PCR primer for the amplification of SEQ ID NO: 22.

SEQ ID NO: 115 is a forward PCR primer for the amplification of SEQ ID NO: 23.

SEQ ID NO: 116 is a forward PCR primer for the amplification of SEQ ID NO: 23.

SEQ ID NO: 117 is a reverse PCR primer for the amplification of SEQ ID NO: 23.

SEQ ID NO: 118 is a forward PCR primer for the amplification of magi99055.

SEQ ID NO: 119 is a forward PCR primer for the amplification of magi99055.

SEQ ID NO: 120 is a reverse PCR primer for the amplification of magi99055.

SEQ ID NO: 121 is a forward PCR primer for the amplification of SEQ ID NO: 24.

SEQ ID NO: 122 is a forward PCR primer for the amplification of SEQ ID NO: 24.

SEQ ID NO: 123 is a reverse PCR primer for the amplification of SEQ ID NO: 24.

SEQ ID NO: 124 is a forward PCR primer for the amplification of SEQ ID NO: 25.

SEQ ID NO: 125 is a forward PCR primer for the amplification of SEQ ID NO: 25.

SEQ ID NO: 126 is a reverse PCR primer for the amplification of SEQ ID NO: 25.

SEQ ID NO: 127 is a forward PCR primer for the amplification of SEQ ID NO: 26.

SEQ ID NO: 128 is a forward PCR primer for the amplification of SEQ ID NO: 26.

SEQ ID NO: 129 is a reverse PCR primer for the amplification of SEQ ID NO: 26.

SEQ ID NO: 130 is a forward PCR primer for the amplification of SEQ ID NO: 27.

SEQ ID NO: 131 is a forward PCR primer for the amplification of SEQ ID NO: 27.

SEQ ID NO: 132 is a reverse PCR primer for the amplification of SEQ ID NO: 27.

SEQ ID NO: 133 is a forward PCR primer for the amplification of SEQ ID NO: 28.

SEQ ID NO: 134 is a forward PCR primer for the amplification of SEQ ID NO: 28.

SEQ ID NO: 135 is a reverse PCR primer for the amplification of SEQ ID NO: 28.

SEQ ID NO: 136 is a forward PCR primer for the amplification of SEQ ID NO: 29.

SEQ ID NO: 137 is a forward PCR primer for the amplification of SEQ ID NO: 29.

SEQ ID NO: 138 is a reverse PCR primer for the amplification of SEQ ID NO: 29.

SEQ ID NO: 139 is a forward PCR primer for the amplification of PHM13823.7.

SEQ ID NO: 140 is a forward PCR primer for the amplification of PHM13823.7.

SEQ ID NO: 141 is a reverse PCR primer for the amplification of PHM13823.7.

SEQ ID NO: 142 is a forward PCR primer for the amplification of PHM2324.23.

SEQ ID NO: 143 is a forward PCR primer for the amplification of PHM2324.23.

SEQ ID NO: 144 is a reverse PCR primer for the amplification of PHM2324.23.

SEQ ID NO: 145 is a forward PCR primer for the amplification of PZA03070.9.

SEQ ID NO: 146 is a forward PCR primer for the amplification of PZA03070.9.

SEQ ID NO: 147 is a reverse PCR primer for the amplification of PZA03070.9.

SEQ ID NO: 148 is a forward PCR primer for the amplification of PZA00279.2.

SEQ ID NO: 149 is a forward PCR primer for the amplification of PZA00279.2.

SEQ ID NO: 150 is a reverse PCR primer for the amplification of PZA00279.2.

SEQ ID NO: 151 is a forward PCR primer for the amplification of SEQ ID NO: 30.

SEQ ID NO: 152 is a forward PCR primer for the amplification of SEQ ID NO: 30.

SEQ ID NO: 153 is a reverse PCR primer for the amplification of SEQ ID NO: 30.

SEQ ID NO: 154 is a forward PCR primer for the amplification of SEQ ID NO: 31.

SEQ ID NO: 155 is a forward PCR primer for the amplification of SEQ ID NO: 31.

SEQ ID NO: 156 is a reverse PCR primer for the amplification of SEQ ID NO: 31.

SEQ ID NO: 157 is a forward PCR primer for the amplification of SEQ ID NO: 32.

SEQ ID NO: 158 is a forward PCR primer for the amplification of SEQ ID NO: 32.

SEQ ID NO: 159 is a reverse PCR primer for the amplification of SEQ ID NO: 32.

SEQ ID NO: 160 is a forward PCR primer for the amplification of SEQ ID NO: 33.

SEQ ID NO: 161 is a forward PCR primer for the amplification of SEQ ID NO: 33.

SEQ ID NO: 162 is a reverse PCR primer for the amplification of SEQ ID NO: 33.

SEQ ID NO: 163 is a forward PCR primer for the amplification of magi52178.

SEQ ID NO: 164 is a forward PCR primer for the amplification of magi52178.

SEQ ID NO: 165 is a reverse PCR primer for the amplification of magi52178.

SEQ ID NO: 166 is a forward PCR primer for the amplification of SEQ ID NO: 34.

SEQ ID NO: 167 is a forward PCR primer for the amplification of SEQ ID NO: 34.

SEQ ID NO: 168 is a reverse PCR primer for the amplification of SEQ ID NO: 34.

SEQ ID NO: 169 is a forward PCR primer for the amplification of PHM6428.11.

SEQ ID NO: 170 is a forward PCR primer for the amplification of PHM6428.11.

SEQ ID NO: 171 is a reverse PCR primer for the amplification of PHM6428.11.

SEQ ID NO: 172 is a forward PCR primer for the amplification of PZA01470.1.

SEQ ID NO: 173 is a forward PCR primer for the amplification of PZA01470.1.

SEQ ID NO: 174 is a reverse PCR primer for the amplification of PZA01470.1.

SEQ ID NO: 175 is a forward PCR primer for the amplification of SEQ ID NO: 35.

SEQ ID NO: 176 is a forward PCR primer for the amplification of SEQ ID NO: 35.

SEQ ID NO: 177 is a reverse PCR primer for the amplification of SEQ ID NO: 35.

SEQ ID NO: 178 is a forward PCR primer for the amplification of SEQ ID NO: 36.

SEQ ID NO: 179 is a forward PCR primer for the amplification of SEQ ID NO: 36.

SEQ ID NO: 180 is a reverse PCR primer for the amplification of SEQ ID NO: 36.

SEQ ID NO: 181 is a forward PCR primer for the amplification of SEQ ID NO: 37.

SEQ ID NO: 182 is a forward PCR primer for the amplification of SEQ ID NO: 37.

SEQ ID NO: 183 is a reverse PCR primer for the amplification of SEQ ID NO: 37.

SEQ ID NO: 184 is a forward PCR primer for the amplification of SEQ ID NO: 38.

SEQ ID NO: 185 is a forward PCR primer for the amplification of SEQ ID NO: 38.

SEQ ID NO: 186 is a reverse PCR primer for the amplification of SEQ ID NO: 38.

SEQ ID NO: 187 is a forward PCR primer for the amplification of SEQ ID NO: 39.

SEQ ID NO: 188 is a forward PCR primer for the amplification of SEQ ID NO: 39.

SEQ ID NO: 189 is a reverse PCR primer for the amplification of SEQ ID NO: 39.

SEQ ID NO: 190 is a forward PCR primer for the amplification of PZA00466.1.

SEQ ID NO: 191 is a forward PCR primer for the amplification of PZA00466.1.

SEQ ID NO: 192 is a reverse PCR primer for the amplification of PZA00466.1.

SEQ ID NO: 193 is a forward PCR primer for the amplification of PZA01272.1.

SEQ ID NO: 194 is a forward PCR primer for the amplification of PZA01272.1.

SEQ ID NO: 195 is a reverse PCR primer for the amplification of PZA01272.1.

SEQ ID NO: 196 is a forward PCR primer for the amplification of PZB01899.1.

SEQ ID NO: 197 is a forward PCR primer for the amplification of PZB01899.1.

SEQ ID NO: 198 is a reverse PCR primer for the amplification of PZB01899.1.

SEQ ID NO: 199 is a forward PCR primer for the amplification of PZB01963.1.

SEQ ID NO: 200 is a forward PCR primer for the amplification of PZB01963.1.

SEQ ID NO: 201 is a reverse PCR primer for the amplification of PZB01963.1.

### DETAILED DESCRIPTION OF THE INVENTION

The present invention provides methods for identifying and selecting maize plants with decreased flowering time. The following definitions are provided as an aid to understand the invention.

The term “allele” refers to one of two or more different nucleotide sequences that occur at a specific locus.

An “amplicon” is amplified nucleic acid, e.g., a nucleic acid that is produced by amplifying a template nucleic acid by any available amplification method (e.g., PCR, LCR, transcription, or the like).

The term “amplifying” in the context of nucleic acid amplification is any process whereby additional copies of a selected nucleic acid for a transcribed form thereof) are produced. Typical amplification methods include various polymerase based replication methods, including the polymerase chain reaction (PCR), ligase mediated methods such as the ligase chain reaction (LCR) and RNA polymerase based amplification (e.g., by transcription) methods.

An allele is “associated with” a trait when it is linked to it and when the presence of the allele is an indicator that the desired trait or trait form will occur in a plant comprising the allele. The “B73 reference genome, version 2” is the physical and genetic framework of the maize B73 genome. It is the result of a sequencing effort utilizing a minimal tiling path of approximately 19,000 mapped BAC clones, and focusing on producing high-quality sequence coverage of all identifiable gene-containing regions of the maize genome. These regions were ordered, oriented, and along with all of the intergenic sequences, anchored to the extant physical and genetic maps of the maize genome. It can be accessed using a genome browser, the Maize Genome Browser, that is publicly available on the internet that facilitates user interaction with sequence and map data.

A “bacterial artificial chromosome (BAC)” is a cloning vector derived from the naturally occurring F factor of Escherichia coli. BACs can accept large inserts of DNA sequence. In maize, a number of BACs, or bacterial artificial chromosomes, each containing a large insert of maize genomic DNA, have been assembled into contigs (overlapping contiguous genetic fragments, or “contiguous DNA”).

“Backcrossing” refers to the process whereby hybrid progeny are repeatedly crossed back to one of the parents. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. (1995) Marker-assisted backcrossing: a practical example, in Techniques et Utilisations des Marqueurs Moleculaires Les Colloques, Vol. 72, pp. 45-56, and Openshaw et al., (1994) Marker-assisted Selection in Backcross Breeding, Analysis of Molecular Marker Data, pp. 41-43. The initial cross gives rise to the F₁ generation: the term “BC1” then refers to the second use of the recurrent parent, “BC2” refers to the third use of the recurrent parent, and so on.

A centimorgan (“cM”) is a unit of measure of recombination frequency. One cM is equal to a 1% chance that a marker at one genetic locus will be separated from a marker at a second locus due to crossing over in a single generation.

The term “complement” refers to a nucleotide sequence that is complementary to a given nucleotide sequence, i.e., the sequences are related by the base-pairing rules.

The term “contiguous DNA” refers to overlapping contiguous genetic fragments.

The term “crossed” or “cross” means the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

“Days to anthesis” refers to the number of days from the planting date when 50% of the plants are shedding pollen.

“Days to silking” refers to the number of days from the planting when 50% of the plants have extruded silk.

The term “dent” refers to a field corn kernel type that has soft cores of starch which cause the end of the kernels to collapse or dent during drying.

As used herein, an “elite line” is any line that has resulted from breeding and selection for superior agronomic performance.

A “favorable allele” is the allele at a particular locus that confers, or contributes to, a desirable phenotype, e.g., decreased flowering time, or alternatively, is an allele that allows the identification of plants with increased flowering time that can be removed from a breeding program or planting (“counterselection”). A favorable allele of a marker is a marker allele that segregates with the favorable phenotype, or alternatively, segregates with the unfavorable plant phenotype, therefore providing the benefit of identifying plants.

“Fragment” is intended to mean a portion of a nucleotide sequence. Fragments can be used as hybridization probes or PCR primers using methods disclosed herein.

The term “flint” refers to a field corn kernel type that has mostly hard, glassy endosperm with smooth, hard seed coats.

“Flowering time” refers to the number of days to flower from planting. Time to flowering is measured as “days to antithesis” and/or “days to silking.”

“Growing degree units (GDU)” refers to the number of heat units accumulated over time calculated using the Barger Method, wherein the heat units for each 24-hour period are: [(Max. temp+Min. temp)/2]−50, with 86° F. the highest maximum temperature used and 50° F. the lowest minimum temperature used. A “genetic map” is a description of genetic linkage relationships among loci on one or more chromosomes (or chromosomes) within a given species, generally depicted in a diagrammatic or tabular form. For each genetic map, distances between loci are measured by the recombination frequencies between them, and recombinations between loci can be detected using a variety of molecular genetic markers (also called molecular markers).

A genetic map is a product of the mapping population, types of markers used, and the polymorphic potential of each marker between different populations. The order and genetic distances between loci can differ from one genetic map to another. However, information such as marker position and order can be correlated between maps by determining the physical location of the markers on the chromosome of interest, using the B73 reference genome, version 2, which is publicly available on the internet. One of ordinary skill in the art can use the publicly available genome browser to determine the physical location of markers on a chromosome.

The term “Genetic Marker” shall refer to any type of nucleic acid based marker, including but not limited to, Restriction Fragment Length Polymorphism (RFLP) (Botstein et al, 1998), Simple Sequence Repeat (SSR) (Jacob et al., 1991), Random Amplified Polymorphic DNA (RAPD) (Welsh et al., 1990), Cleaved Amplified Polymorphic Sequences (CAPS) (Rafalski and Tingey, 1993, Trends in Genetics 9:275-280), Amplified Fragment Length Polymorphism (AFLP) (Vos et al, 1995, Nucleic Acids Res. 23:4407-4414), Single Nucleotide Polymorphism (SNP) (Brookes, 1999, Gene 234:177-186), Sequence Characterized Amplified Region (SCAR) (Pecan and Michelmore, 1993, Theor. Appl. Genet, 85:985-993), Sequence Tagged Site (STS) (Onozaki et al. 2004, Euphytica 138:255-262), Single Stranded Conformation Polymorphism (SSCP) (Orita et al., 1989, Proc Natl Aced Sci USA 86:2766-2770). Inter-Simple Sequence Repeat (ISR) (Blair et al. 1999, Theor. Appl. Genet. 98:780-792), Inter-Retrotransposon Amplified Polymorphism (IRAP), Retrotransposon-Microsatellite Amplified Polymorphism (REMAP) (Kalendar et al., 1999, Theor. Appl. Genet 98:704-711), an RNA cleavage product (such as a Lynx tag), and the like.

“Genetic recombination frequency” is the frequency of a crossing over event (recombination) between two genetic loci. Recombination frequency can be observed by following the segregation of markers and/or traits following meiosis.

“Genome” refers to the total DNA, or the entire set of genes, carried by a chromosome or chromosome set.

The term “genotype” is the genetic constitution of an individual (or group of individuals) at one or more genetic loci, as contrasted with the observable trait (the phenotype). Genotype is defined by the allele(s) of one or more known loci that the individual has inherited from its parents. The term genotype can be used to refer to an individual's genetic constitution at a single locus, at multiple led, or, more generally, the term genotype can be used to refer to an individual's genetic make-up for all the genes in its genome.

“Germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leafs, stems, pollen, or cells that can be cultured into a whole plant.

A “haplotype” is the genotype of an individual at a plurality of genetic loci, i.e. a combination of alleles. Typically, the genetic loci described by a haplotype are physically and genetically linked, i.e., on the same chromosome segment. The term “haplotype” can refer to sequence, polymorphisms at a particular locus, such as a single marker locus, or sequence polymorphisms at multiple loci along a chromosomal segment in a given genome. The former can also be referred to as “marker haplotypes” or “marker alleles”, while the latter can be referred to as “long-range haplotypes”.

A “heterotic group” comprises a set of genotypes that perform well when crossed with genotypes from a different heterotic group (Hallauer at al. (1998) Corn breeding, p. 463-564. In G. F. Sprague and J. W. Dudley (ed) Corn and corn improvement). Inbred lines are classified into heterotic groups, and are further subdivided into families within a heterotic group, based on several criteria such as pedigree, molecular marker-based associations, and performance in hybrid combinations (Smith at al. (1990) Theor. Appl. Gen. 80:833-840). The two most widely used heterotic groups in the United States are referred to as “Iowa Stiff Stalk Synthetic” (BSSS) and “Lancaster” or “Lancaster Sure Crop” (sometimes referred to as NSS, or Iron-Stiff Stalk).

The term “heterozygous” means a genetic condition wherein different alleles reside at corresponding loci on homologous chromosomes.

The term “homozygous” means a genetic condition wherein identical alleles reside at corresponding loci on homologous chromosomes.

The term “hybrid” means a progeny of mating between at least two genetically dissimilar parents. Without limitation, examples of mating schemes include single crosses, modified single cross, double modified single cross, three-way cross, modified three-way cross, and double cross wherein at least one parent in a modified cross is the progeny of a cross between sister lines.

“Hybridization” or “nucleic acid hybridization” refers to the pairing of complementary RNA and DNA strands as well as the pairing of complementary DNA single strands.

The term “hybridize” means the formation of base pairs between complementary regions of nucleic acid strands.

The term “inbred” means a line that has been bred for genetic homogeneity.

The term “indel” refers to an insertion or deletion, wherein one line may be referred to as having an insertion relative to a second line, or the second line may be referred to as having a deletion relative to the first line.

The term “introgression” or “introgressing” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., a selected allele of a marker, a QTL, a transgene, or the like. In any case, offspring comprising the desired allele can be repeatedly backcrossed to a line having a desired genetic background and selected for the desired allele, to result in the allele becoming fixed in a selected genetic background. For example, the chromosome 1 locus described herein may be introgressed into a recurrent parent that has a later flowering time. The recurrent parent line with the introgressed gene or locus then has decreased flowering time.

As used herein, the term “linkage” is used to describe the degree with which one marker locus is associated with another marker locus or some other locus (for example, a flowering time locus). The linkage relationship between a molecular marker and a phenotype is given as a “probability” or “adjusted probability”. Linkage can be expressed as a desired limit or range. For example, in some embodiments, any marker is linked (genetically and physically) to any other marker when the markers are separated by less than 50, 40, 30, 25, 20, or 15 map units for cM). In some aspects, it is advantageous to define a bracketed range of linkage, for example, between 10 and 20 cM, between 10 and 30 cM, or between 10 and 40 cM. The more closely a marker is linked to a second locus, the better an indicator for the second locus that marker becomes. Thus, “closely linked loci” such as a marker locus and a second locus display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10 (e.g., about 9%, 8%, 7%, 6%, 5%, 4%, 3%, 2%, 1%, 0.75%, 0.5%, 0.25%, or less) are also said to be “proximal to” each other. Since one cM is the distance between two markers that show a 1% recombination frequency, any marker is closely linked (genetically and physically) to any other marker that is in close proximity, e.g., at or less than 10 cM distant. Two closely linked markers on the same chromosome can be positioned 9, 8, 7, 6, 5, 4, 3, 2, 1, 0.75, 0.5 or 0.25 cM or less from each other.

The term “linkage disequilibrium” refers to a non-random segregation of genetic loci or traits for both). In either case, linkage disequilibrium implies that the relevant loci are within sufficient physical proximity along a length of a chromosome so that they segregate together with greater than random (i.e., non-random) frequency (in the case of co-segregating traits, the loci that underlie the traits are in sufficient proximity to each other). Markers that show linkage disequilibrium are considered linked. Linked loci co-segregate more than 50% of the time, e.g., from about 51% to about 100% of the time. In other words, two markers that co-segregate have a recombination frequency of less than 50% (and by definition, are separated by less than 50 cM on the same chromosome.) As used herein, linkage can be between two markers, or alternatively between a marker and a phenotype. A marker locus can be “associated with” (linked to) a trait, e.g., decreased flowering time. The degree of linkage of a molecular marker to a phenotypic trait is measured, e.g. as a statistical probability of co-segregation of that molecular marker with the phenotype.

Linkage disequilibrium is most commonly assessed using the measure r², which is calculated using the formula described by Hill, W. G. and Robertson, A, Theor Appl. Genet 38:226-231 (1988). When r²=1, complete LD exists between the two marker loci, meaning that the markers have not been separated by recombination and have the same allele frequency. Values for r² above ⅓ indicate sufficiently strong LD to be useful for mapping (Ardlie at al., Nature Reviews Genetics 3:299-309 (2002)). Hence, alleles are in linkage disequilibrium when r² values between pairwise marker loci are greater than or equal to 0.33, 0.4, 0.5, 0.6, 0.7, 0.8, 0.9, or 1.0.

As used herein, “linkage equilibrium” describes a situation where two markers independently segregate, i.e., sort among progeny randomly. Markers that show linkage equilibrium are considered unlinked (whether or not they lie on the same chromosome).

A “locus” is a position on a chromosome where a gene or marker is located.

“Maize” refers to a plant of the Zea mays L. ssp. mays and is also known as “corn”.

The term “maize plant” includes: whole maize plants, maize plant cells, maize plant protoplast, maize plant cell or maize tissue cultures from which maize plants can be regenerated, maize plant calli, and maize plant cells that are intact in maize plants or parts of maize plants, such as maize seeds, maize cobs, maize flowers, maize cotyledons, maize leaves, maize stems, maize buds, maize roots, maize root tips, and the like.

A “marker” is a nucleotide sequence or encoded product thereof (e.g., a protein) used as a point of reference. For markers to be useful at detecting recombinations, they need to detect differences, or polymorphisms, within the population being monitored. For molecular markers, this means differences at the DNA level due to polynucleotide sequence differences (e.g. SSRs, RFLPs, AFLPs, SNPs). The genomic variability can be of any origin, for example, insertions, deletions, duplications, repetitive elements, point mutations, recombination events, or the presence and sequence of transposable elements. Molecular markers can be derived from genomic or expressed nucleic acids (e.g., ESTs) and can also refer to nucleic acids used as probes or primer pairs capable of amplifying sequence fragments via the use of PCR-based methods. A large number of maize molecular markers are known in the art, and are published or available from various sources, such as the Maize GDB Internet resource and the Arizona Genomics Institute Internet resource run by the University of Arizona.

Markers corresponding to genetic polymorphisms between members of a population can be detected by methods well-established in the art. These include, e.g., DNA sequencing, PCR-based sequence specific amplification methods, detection of RFLPs, detection of isozyme markers, detection of polynucleotide polymorphisms by allele specific hybridization (ASH), detection of amplified variable sequences of the plant genome, detection of self-sustained sequence replication, detection of SSRs, detection of SNPs, or detection of AFLPs. Well established methods are also known for the detection of expressed sequence tags (ESTs) and SSR markers derived from EST sequences and RAPDs.

A “marker allele”, alternatively an “allele of a marker locus”, can refer to one of a plurality of polymorphic nucleotide sequences found at a marker locus in a population that is polymorphic for the marker locus.

“Marker assisted selection” (or MAS) is a process by which phenotypes are selected based on marker genotypes.

“Marker assisted counter-selection” is a process by which marker genotypes are used to identify plants that will not be selected, allowing them to be removed from a breeding program or planting.

A “marker locus” is a specific chromosome location in the genome of a species when a specific marker can be found. A marker locus can be used to track the presence of a second linked locus, e.g., a linked locus that encodes or contributes to expression of a phenotypic trait. For example, a marker locus can be used to monitor segregation of alleles at a locus, such as a QTL or single gene, that are genetically or physically linked to the marker locus.

A “marker probe” is a nucleic add sequence or molecule that can be used to identify the presence of a marker locus, e.g., a nucleic acid probe that is complementary to a marker locus sequence, through nucleic add hybridization. Marker probes comprising 30 or more contiguous nucleotides of the marker locus (“all or a portion” of the marker locus sequence) may be used for nucleic acid hybridization. Alternatively, in some aspects, a marker probe refers to a probe of any type that is able to distinguish (i.e. genotype) the particular allele that is present at a marker locus.

The term “molecular marker” may be used to refer to a genetic marker, as defined above, or an encoded product thereof (e.g., a protein) used as a point of reference when identifying a linked locus. A marker can be derived from genomic nucleotide sequences or from expressed nucleotide sequences (e.g., from a spliced RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “molecular marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. Nucleic acids are “complementary” when they specifically hybridize in solution, e.g., according to Watson-Crick base pairing rules. Some of the markers described herein are also referred to as hybridization markers when located on an indel region, such as the non-collinear region described herein. This is because the insertion region is, by definition, a polymorphism vis a via a plant without the insertion. Thus, the marker need only indicate whether the indel region is present or absent. Any suitable marker detection technology may be used to identify such a hybridization marker, e.g., SNP technology is used in the examples provided herein.

“Nucleotide sequence”, “polynucleotide”, “nucleic acid sequence”, and “nucleic acid fragment” are used interchangeably and refer to a polymer of RNA or DNA that is single- or double-stranded, optionally containing synthetic, non-natural or altered nucleotide bases. A “nucleotide” is a monomeric unit from which DNA or RNA polymers are constructed, and consists of a purine or pyrimidine base, a pentose, and a phosphoric acid group. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate. “U” for uridylate, “T” for deoxythymidylate, “R” for purines (A or G), “Y” for pyrimidines (C or T), “K” for G or T, “H” for A or C or T, “I” for inosine, and “N” for any nucleotide.

The terms “phenotype”, or “phenotypic trait” or “trait” refers to one or more traits of an organism. The phenotype can be observable to the naked eye, or by any other means of evaluation known in the art, e.g., microscopy, biochemical analysis, or an electromechanical assay. In some cases, a phenotype is directly controlled by a single gene or genetic locus, i.e., a “single gene trait”. In other cases, a phenotype is the result of several genes.

A “physical map” of the genome is a map showing the linear order of identifiable landmarks (including genes, markers, etc.) on chromosome DNA. However, in contrast to genetic maps, the distances between landmarks are absolute (for example, measured in base pairs or isolated and overlapping contiguous genetic fragments) and not based on genetic recombination.

A “plant” can be a whole plant, any part thereof, or a cell or tissue culture derived from a plant. Thus, the term “plant” can refer to any of: whole plants, plant components or organs (e.g., leaves, stems, roots, etc.), plant tissues, seeds, plant cells, and/or progeny of the same. A plant cell is a cell of a plant, taken from a plant, or derived through culture from a cell taken from a plant.

A “polymorphism” is a variation in the DNA that is too common to be due merely to new mutation. A polymorphism must have a frequency of at least 1% in a population. A polymorphism can be a single nucleotide polymorphism, or SNP, or an insertion/deletion polymorphism, also referred to herein as an “indel”.

The term “progeny” refers to the offspring generated from a cross.

A “progeny plant” is generated from a cross between two plants.

A “reference sequence” is a defined sequence used as a basis for sequence comparison. The reference sequence is obtained by genotyping a number of lines at the locus, aligning the nucleotide sequences in a sequence alignment program (e.g. Sequencher), and then obtaining the consensus sequence of the alignment.

A “single nucleotide polymorphism (SNP)” is an allelic single nucleotide—A, T, C or G—variation within a DNA sequence representing one locus of at least two individuals of the same species. For example, two sequenced DNA fragments representing the same locus from at least two individuals of the same species, AAGCCTA to AAGCTTA, contain a difference in a single nucleotide.

The “Stiff Stalk” heterotic group represents a major heterotic group in the northern U.S. and Canadian corn growing regions. It can also be referred to as the Iowa Stiff Stalk Synthetic for BSSS heterotic group.

A “topeross test” is a progeny test derived by crossing each parent with the same tester, usually a homozygous line. The parent being tested can be an open-pollinated variety, a cross, or an inbred line.

The term “quantitative trait locus (QTL)” means a locus that controls to some degree numerically representable traits that are usually continuously distributed.

Before describing the present invention in detail, it should be understood that this invention is not limited to particular embodiments. It also should be understood that the terminology used herein is for the purpose of describing particular embodiments, and is not intended to be limiting. As used herein and in the appended claims, terms in the singular and the singular forms “a”, “an” and “the”, for example, include plural referents unless the content clearly dictates otherwise. Thus, for example, reference to “plant”, “the plant” or “a plant” also includes a plurality of plants. Depending on the context, use of the term “plant” can also include genetically similar or identical progeny of that plant. The use of the term “a nucleic acid” optionally includes many copies of that nucleic acid molecule.

Genetic Mapping. It has been recognized for quite some time that specific genetic loci correlating with particular phenotypes, such as reduced flowering time, can be mapped in an organism's genome. The plant breeder can advantageously use molecular markers to identify desired individuals by detecting marker alleles that show a statistically significant probability of co-segregation with a desired phenotype, manifested as linkage disequilibrium. By identifying a molecular marker or clusters of molecular markers that co-segregate with a trait of interest, the breeder is able to rapidly select a desired phenotype by selecting for the proper molecular marker allele (a process called marker-assisted selection, or MAS).

A variety of methods well known in the art are available for detecting molecular markers or clusters of molecular markers that co-segregate with a trait of interest, such as decreased flowering time. The basic idea underlying these methods is the detection of markers, for which alternative genotypes (or alleles) have significantly different average phenotypes. Thus, one makes a comparison among marker loci of the magnitude of difference among alternative genotypes (or alleles) or the level of significance of that difference. Trait genes are inferred to be located nearest the marker(s) that have the greatest associated genotypic difference.

Two such methods used to detect trait loci of interest are: 1) Population-based association analysis and 2) Traditional linkage analysis. In a population-based association analysis, lines are obtained from pre-existing populations with multiple founders, e.g. elite breeding lines. Population-based association analyses rely on the decay of linkage disequilibrium (LD) and the idea that in an unstructured population, only correlations between genes controlling a trait of interest and markers closely linked to those genes will remain after so many generations of random mating. In reality, most pre-existing populations have population substructure. Thus, the use of a structured association approach helps to control population structure by allocating individuals to populations using data obtained from markers randomly distributed across the genome, thereby minimizing disequilibrium due to population structure within the individual populations (also called subpopulations). The phenotypic values are compared to the genotypes (alleles) at each, marker locus for each line in the subpopulation. A significant marker-trait association indicates the dose proximity between the marker locus and one or more genetic loci that are involved in the expression of that trait.

The same principles underlie traditional linkage analysis; however, LD is generated by creating a population from a small number of founders. The founders are selected to maximize the level of polymorphism within the constructed population, and polymorphic sites are assessed for their level of cosegregation with a given phenotype. A number of statistical methods have been used to identify significant marker-trait associations. One such method is an interval mapping approach (Lander and Botstein, Genetics 121:185-199 (1989), in which each of many positions along a genetic map (say at 1 cM intervals) is tested for the likelihood that a gene controlling a trait of interest is located at that position. The genotype/phenotype data are used to calculate for each test position a LOD score (log of likelihood ratio). When the LOD score exceeds a threshold value, there is significant evidence for the location of a gene controlling the trait of interest at that position on the genetic map (which will fall between two particular marker loci).

Markers Associated with Flowering Time. Markers associated with decreased flowering time are identified herein. The methods involve detecting the presence of at least four marker alleles associated with decreased flowering time in the germplasm of a maize plant. The marker loci can be selected from each of four marker nucleic acid groups (i)-(iv) provided in Table 1: (i) DAS-PZ-10450, DAS-PZ-12076, DAS-PZ-16344, DAS-PZ-1867, DAS-PZ-2256, DAS-PZ-6992, DAS-PZ-8291, DAS-PZ-9937, DSDS-0066-1, KG-2679163, magi18365, Mo17-101121, PZA03746.1, PZA02737.1, and DAS-PZ-6563; (ii) DAS-PZ-10083, DAS-PZ-1255, DAS-PZ-14161, DAS-PZ-15602, DAS-PZ-19469, DAS-PZ-3132, DAS-PZ-4347, DAS-PZ-5837, DAS-PZ-8111, DAS-PZ-880, DAS-PZ-9899, magi99055, Mo17-10988, Mo17-11370, Mo17-11923, Mo17-13135, Mo17-1316, Mo17-13288, PHM13823.7, PHM2324.23, PZA03070.9, and PZA00279.2; (iii) DAS-PZ-2559, DAS-PZ-3168, DAS-PZ-3394, magi104662, magi52178, Mo17-10465, PHM6428.11, PZA01470.1, and DAS-PZ-9978; and, (iv) DAS-PZ-366, KG-2566510, KG-2624948, Mo17-13418, PZA00466.1, PZA01272.1, PZB01899.1, and PZB01963.1, and any other marker linked to these markers (linked markers can be determined from the MaizeGDB and Panzea resources).

A common measure of linkage is the frequency with which traits cosegregate. This can be expressed as a percentage of cosegregation (recombination frequency) or in centiMorgans (cM). The cM is a unit of measure of genetic recombination frequency. One cM is equal to a 1% chance that a trait at one genetic locus will be separated from a trait at another locus due to crossing over in a single generation (meaning the traits segregate together 99% of the time). Because chromosomal distance is approximately proportional to the frequency of crossing over events between traits, there is an approximate physical distance that correlates with recombination frequency.

Marker loci are themselves traits and can be assessed according to standard linkage analysis by tracking the marker loci during segregation. Thus, one cM is equal to a 1% chance that a marker locus will be separated from another locus, due to crossing over in a single generation.

Other markers linked to the markers listed in Table 1 can be used to predict flowering time in a maize plant. This includes any marker within 50 cM of markers within groups (i)-(iv): (i) DAS-PZ-10450, DAS-PZ-12076, DAS-PZ-16344, DAS-PZ-1867, DAS-PZ-2256, DAS-PZ-6992, DAS-PZ-8291, DAS-PZ-9937, DSDS-0066-1, KG-2679163, magi18365, Mo17-101121, PZA03746.1, PZA02737.1, and DAS-PZ-6563; (ii) DAS-PZ-10083, DAS-PZ-1255, DAS-PZ-14161, DAS-PZ-15602, DAS-PZ-19469, DAS-PZ-3132, DAS-PZ-4347, DAS-PZ-5837, DAS-PZ-8111, DAS-PZ-880, DAS-PZ-9899, magi99055, Mo17-10988, Mo17-11370, Mo17-11923, Mo17-13135, Mo17-1316, Mo17-13288, PHM13823.7, PHM2324.23, PZA03070.9, and PZA00279.2; (iii) DAS-PZ-2559, DAS-PZ-3168, DAS-PZ-3394, magi104662, magi52178, Mo17-10465, PHM6428.11, PZA01470.1, and DAS-PZ-9978; and, (iv) DAS-PZ-366, KG-2566510, KG-2624948, Mo17-13418, PZA00466.1, PZA01272.1, PZB01899.1, and PZB01963.1, the markers associated with the flowering time. The closer a marker is to a gene controlling a trait of interest, the more effective and advantageous that marker is as an indicator for the desired trait. Closely linked loci display an inter-locus cross-over frequency of about 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci (e.g., a marker locus and a target locus) display a recombination frequency of about 1% or less, e.g., about 0.75% or less, more preferably about 0.5% or less, or yet more preferably about 0.25% or less. Thus, the loci are about 10 cM, 9 cM, 8 cM, 7 cM, 6 cM, 5 cM, 4 cM, 3 cM, 2 cM, 1 cM, 0.75 cM, 0.5 cM or 0.25 cM or less apart. Put another way, two loci that are localized to the same chromosome, and at such a distance that recombination between the two loci occurs at a frequency of less than 10% (e.g., about 9%, 8% 7%, 6%, 5%, 4%, 3%, 2% 1%, 0.75%, 0.5%, 0.25.degree., or less) are said to be “proximal to” each other.

Although particular marker alleles can show co-segregation with decreased flowering time, it is important to note that the marker locus is not necessarily responsible for the expression of the decreased flowering time phenotype. For example, it is not a requirement that the marker polynucleotide sequence be part of a gene that imparts decreased flowering time (for example, be part of the gene open reading frame). The association between a specific marker allele and the decreased flowering time phenotype is due to the original “coupling” linkage phase between the marker allele and the allele in the ancestral maize line from which the allele originated. Eventually, with repeated recombination, crossing over events between the marker and genetic locus can change this orientation. For this reason, the favorable marker allele may change depending on the linkage phase that exists within the donor parent used to create segregating populations. This does not change the fact that the marker can be used to monitor segregation of the phenotype. It only changes which marker allele is considered favorable in a given segregating population.

The present invention includes isolated nucleic acid molecules. Such molecules include those nucleic acid molecules capable of detecting a polymorphism genetically or physically linked to a flowering time locus. Such molecules can be referred to as markers. Additional markers can be obtained that are linked to flowering time locus i, ii, iii, or iv by available techniques. In one aspect, the nucleic acid molecule is capable of detecting the presence or absence of a marker located less than 30, 20, 10, 5, 2, or 1 cM from a flowering time locus. In another aspect, the nucleic acid molecule is capable of detecting a marker in a locus selected from the group i, ii, iii, or iv. In a further aspect, a nucleic acid molecule is selected from the group consisting of SEQ ID NO: 1 through SEQ ID NO: 201 fragments thereof, complements thereof, and nucleic acid molecules capable of specifically hybridizing to one or more of these nucleic acid molecules. In another aspect, a nucleic acid molecule is selected from the publicly available markers listed in Table 1, fragments thereof, complements thereof, and nucleic acid molecules capable of specifically hybridizing to one or more of these nucleic acid molecules.

A marker of the invention can also be a combination of alleles at marker loci, otherwise known as a haplotype. The skilled artisan would expect that there might be additional polymorphic sites at marker loci in and around the flowering time markers identified herein, wherein one, or more polymorphic sites is in linkage disequilibrium (LD) with an allele associated with decreased flowering time. Two particular alleles at different polymorphic sites are said to be in LD if the presence of the allele at one of the sites tends to predict the presence of the allele at the other site on the same chromosome (Stevens, Mol. Diag. 4:309-17 (1999)). Marker Assisted Selection.

Molecular markers can be used in a variety of, plant breeding applications (e.g. see Staub et al. (1996) Hortscience 729-741; Tanksley (1983) Plant Molecular Biology Reporter 1: 3-8). One of the main areas of interest is to increase the efficiency of backcrossing and introgressing genes using marker-assisted selection (MAS). A molecular marker that demonstrates linkage with a locus affecting a desired phenotypic trait provides a useful tool for the selection of the trait in a plant population. This is particularly true with traits that are difficult to phenotype due to their dependence on environmental conditions. This category includes traits related to the resistance to biotic and abiotic stresses. This category also includes traits that are very expensive to phenotype because of laborious artificial inoculation or maintenance of managed stress environments. Another category of traits includes those which are associated with destruction of plant per se. Destructive phenotyping has been a bottleneck to implement MAS for the seed quality traits. Because DNA marker assays are not environmentally dependent, are robust, reliable, less laborious, less costly and take up less physical space than field phenotyping, much larger populations can be assayed, increasing the chances of finding a recombinant with the target segment from the donor line moved to the recipient line. The closer the linkage, the more useful the marker, as recombination is less likely to occur between the marker and the gene causing the trait, which can result in false positives. Having flanking markers decreases the chances that false positive selection will occur as a double recombination event would be needed. The ideal situation is to have a marker in the gene itself, so that recombination cannot occur between the marker and the gene. Such a marker is called a ‘perfect marker’.

When a gene is introgressed by MAS, it is not only the gene that is introduced but also the flanking regions (Gepts. (2002). Crop Sci; 42: 1780-1790). This is referred to as “linkage drag.” In the case where the donor plant is highly unrelated to the recipient plant, these flanking regions carry additional genes that may code for agronomically undesirable traits. This “linkage drag” may also result in reduced yield or other negative agronomic characteristics even after multiple cycles of backcrossing into the elite maize line. This is also sometimes referred to as “yield drag.” The size of the flanking region can be decreased by additional backcrossing, although this is not always successful, as breeders do not have control over the size of the region or the recombination breakpoints (Young et al. (1998) Genetics 120:579-585). In classical breeding it is usually only by chance that recombinations are selected that contribute to a reduction in the size of the donor segment (Tanksley et al. (1989). Biotechnology 7: 257-264). Even after 20 backcrosses in backcrosses of this type, one may expect to find a sizeable piece of the donor chromosome still linked to the gene being selected. With markers however, it is possible to select those rare individuals that have experienced recombination near the gene of interest. In 150 backcross plants, there is a 95% chance that at least one plant will have experienced a crossover within 1 cM of the gene, based on a single meiosis map distance. Markers will avow unequivocal identification of those individuals. With one additional backcross of 300 plants, there would be a 95% chance of a crossover within 1 cM single meiosis map distance of the other side of the gene, generating a segment around the target gene of less than 2 cM based on a single meiosis map distance. This can be accomplished in two generations with, markers, while it would have required on average 100 generations without markers (See Tanksley et al., supra). When the exact location of a gene is known, flanking markers surrounding the gene can be utilized to select for recombinations in different population sizes. For example, in smaller population sizes, recombinations may be expected further away from the gene, so more distal flanking markers would be required to detect the recombination.

The availability of the B73 reference genome, version 2 and the integrated linkage maps of the maize genome containing increasing densities of public maize markers, has facilitated maize genetic mapping and MAS. See, e.g. the IBM2 Neighbors maps, which are available online on the Maize GDB website.

The key components to the implementation of MAS are (i) Defining the population within which the marker-trait association will be determined, which can be a segregating population, or a random or structured population; (ii) monitoring the segregation or association of polymorphic markers relative to the trait, and determining linkage or association using statistical methods; (iii) defining a set of desirable markers based on the results of the statistical analysis, and (iv) the use and/or extrapolation of this information to the current set of breeding germplasm to enable marker-based selection decisions to be made. The markers described in this disclosure, as well as other marker types such as SSRs and FLPs, can be used in marker assisted selection protocols.

SSRs can be defined as relatively short runs of tandemly repeated DNA with lengths of 6 bp or less (Tautz (1989) Nucleic Acid Research 17: 6463-6471; Wang et al. (1994) Theoretical and Applied Genetics, 88:1-6). Polymorphisms arise due to variation in the number of repeat units, probably caused by slippage during DNA replication (Levinson and Gutman (1987) Mol Biol Evol 4: 203-221). The variation in repeat length may be detected by designing PCR primers to the conserved non-repetitive flanking regions (Weber and May (1989) Am J Hum Genet. 44:388-396), SSRs are highly suited to mapping and MAS as they are multi-allelic, codominant, reproducible and amenable to high throughput automation (Rafalski et al. (1996) Generating and using DNA markers in plants. In Non-mammalian genomic analysis: a practical guide. Academic Press, pp 75-135).

Various types of SSR markers can be generated, and SSR profiles from resistant lines can be obtained by gel electrophoresis of the amplification products. Scoring of marker genotype is based on the size of the amplified fragment. An SSR service for maize is available to the public on a contractual basis by DNA Landmarks in Saint-Jean-sur-Richelieu, Quebec, Canada.

Various types of FLP markers can also be generated. Most commonly, amplification primers are used to generate fragment length polymorphisms. Such FLP markers are in many ways similar to SSR markers, except that the region amplified by the primers is not typically a highly repetitive region. Still, the amplified region, or amplicon, will have sufficient variability among germplasm, often due to insertions or deletions, such that the fragments generated by the amplification primers can be distinguished among polymorphic individuals, and such indels are known to occur frequently in maize (Bhattramakki et al. (2002). Plant Mol Biol 48, 539-547; Rafalski (2002b), supra).

SNP markers detect single base pair nucleotide substitutions. Of all the molecular marker types, SNPs are the most abundant, thus having the potential to provide the highest genetic map resolution (Bhattramakki et al. 2002 Plant Molecular Biology 48:539-547). SNPs can be assayed at an even higher level of throughput than SSRs, in a so-called ‘ultra-high-throughput’ fashion, as they do not require large amounts of DNA and automation of the assay may be straight-forward. SNPs also have the promise of being relatively low-cost systems. These three factors together make SNPs highly attractive for use in MAS. Several methods are available for SNP genotyping, including but not limited to, hybridization, primer extension, oligonucleotide ligation, nuclease cleavage, minisequencing and coded spheres. Such methods have been reviewed in: Gut (2001) Hum Mutat 17 pp, 475-492: Shi (2001) Clin Chem 47, pp. 164-172; Kwok (2000) Pharmacogenomics 1, pp. 95-100: Bhattramakki and Rafalski (2001) Discovery and application of single nucleotide polymorphism markers in plants. In: R, J Henry, Ed, Plant Genotyping: The DNA Fingerprinting of Plants, CABI Publishing, VVallingford. A wide range of commercially available technologies utilize these and other methods to interrogate SNPs including Masscode™. (Qiagen), Invader® (Third Wave Technologies), SnapShot® (Applied Biosystems), Taqman® (Applied Biosystems) and Beadarrays™ (Illumina).

A number of SNPs together within a sequence, or across linked sequences, can be used to describe a haplotype for any particular genotype (Ching et al. (2002), BMC Genet. 3:19 pp Gupta et al. 2001, Rafalski (2002b), Plant Science 162:329-333). Haplotypes can be more informative than, single SNPs and can be more descriptive of any particular genotype. For example, single SNP may be allele ‘T’ for a specific line or variety with increased GS tolerance, but the allele ‘T’ might also occur in the maize breeding population being utilized for recurrent parents. In this case, a haplotype, e.g. a combination of alleles at linked SNP markers, may be more informative. Once a unique haplotype has been assigned to a donor chromosomal region, that haplotype can be used in that population or any subset thereof to determine whether an individual has a particular gene. See, for example, WO2003054229. Using automated high throughput marker detection platforms known to those of ordinary skill in the art makes this process highly efficient and effective.

The sequences listed in Table 1 can be readily used to obtain additional polymorphic SNPs (and other markers) linked to the markers listed in this disclosure that are associated with flowering time QTLs. Markers listed in Table 1 can be hybridized to BACs or other genomic libraries, or electronically aligned with genome sequences, to find new sequences in the same approximate location as the described markers.

In addition to SSRs, RFLPs and SNPs, as described above, other types of molecular markers are also widely used, including but not limited to, markers derived from EST sequences, RAPDs, and other nucleic acid based markers.

Isozyme profiles and linked morphological characteristics can, in some cases, also be indirectly used as markers. Even though they do not directly detect DNA differences, they are often influenced by specific genetic differences. However, markers that detect DNA variation are far more numerous and polymorphic than isozyme or morphological markers (Tanksley (1983) Plant Molecular Biology Reporter 1:3-8).

Sequence alignments or contigs may also be used to find sequences upstream or downstream of the specific markers listed herein. These new sequences, close to the markers described herein, are then used to discover and develop functionally equivalent markers. For example, different physical and/or genetic maps are aligned to locate equivalent markers not described within this disclosure but that are within similar regions. These maps may be within the maize species, or even across other species whose genomes share some level of colinearity at macro- and micro-level with maize, such as rice and sorghum.

In general, MAS uses polymorphic markers that have been identified as having a significant likelihood of co-segregation with decreased flowering time. Such markers are presumed to map near quantitative trait loci (QTL), give the plant its decreased flowering time phenotype, and are considered indicators, or markers, for the desired trait. Markers test maize plants for the presence of a desired allele, and those which contain a desired genotype at one or more loci are expected to transfer the desired genotype, along with a desired phenotype, to their progeny. The means to identify maize plants that have decreased flowering time by identifying plants that have a specified allele at from each of the four marker nucleic acid groups (i)-(iv) described herein, including: (i) DAS-PZ-10450, DAS-PZ-12076, DAS-PZ-16344, DAS-PZ-1867, DAS-PZ-2256, DAS-PZ-6992, DAS-PZ-8291, DAS-PZ-9937, DSDS-0066-1, KG-2679163, magi18365, Mo17-101121, PZA03746.1, PZA02737.1, and DAS-PZ-6563; (ii) DAS-PZ-10083, DAS-PZ-1255, DAS-PZ-14161, DAS-PZ-15602, DAS-PZ-19469, DAS-PZ-3132, DAS-PZ-4347, DAS-PZ-5837, DAS-PZ-8111, DAS-PZ-880, DAS-PZ-9899, magi99055, Mo17-10988, Mo17-11370, Mo17-11923, Mo17-13135, Mo17-1316, Mo17-13288, PHM13823.7, PHM2324.23, PZA03070.9, and PZA00279.2; (iii) DAS-PZ-2559, DAS-PZ-3168, DAS-PZ-3394, magi104662, magi52178, Mo17-10465, PHM6428.11, PZA01470.1, and DAS-PZ-9978; and, (iv) DAS-PZ-366, KG-2566510, KG-2624948, Mo17-13418, PZA00466.1, PZA01272.1, PZB01899.1, and PZB01963.1, are presented herein.

The four marker nucleic acid groups presented herein finds use in MAS to select plants that demonstrate decreased flowering time. Any marker that listed in Table 1 can be used for this purpose. In addition, haplotypes comprising alleles at one or more marker loci within each of the four marker nucleic acid groups (i)-(iv) can be used to introduce a decreased flowering time trait into maize lines or varieties. Any allele or haplotype that is in linkage disequilibrium with an allele associated with decreased flowering time can be used in MAS to select plants with decreased flowering time.

### EXAMPLES

The following examples are offered to illustrate, but not to limit, the appended claims. It is understood that the examples and embodiments described herein are for illustrative purposes only and that persons skilled in the art will recognize various reagents or parameters that can be altered without departing from the spirit of the invention or the scope of the appended claims.

### Example 1

Marker framework and use for MAS. A set of common markers can be used to establish a framework for identifying markers linked to a QTL. Closely linked markers flanking the locus of interest that have alleles in linkage disequilibrium with a favorable allele at that locus may be effectively used to select for progeny plants with decreased flowering time. Thus, the markers described in herein, such as those listed in Table 1, as well as other markers genetically or physically mapped to the same chromosomal segment, may be used to select for maize plants with decreased flowering time. Typically, a set of these markers will be used (e.g. 2 or more, 3 or more, 4 or more, 5 or more) in the regions flanking the locus of interest. Optionally, a marker within the actual gene and/or locus may be used. Exemplary primers for amplifying and detecting genomic regions associated with decreased flowering time are given in Table 2.

TABLE 1
Summary of SNP markers associated with decreased
flowering time.

| | | SEQ ID | | Donor |
|---|---|---|---|---|
| Marker | Group | NO | SNP | Allele |
| DAS-PZ-10450 | I | 1 | A/T | TT |
| DAS-PZ-12076 | I | 2 | G/T | TT |
| DAS-PZ-16344 | I | 3 | A/C | CC |
| DAS-PZ-1867 | I | 4 | G/T | TT |
| DAS-PZ-2256 | I | 5 | A/G | GG |
| DAS-PZ-6992 | I | 6 | C/A | AA |
| DAS-PZ-8291 | I | 7 | A/T | TT |
| DAS-PZ-9937 | I | 8 | C/A | AA |
| DSDS-0066-1 | I | 9 | C/T | TT |
| KG-2679163 | I | 10 | C/T | TT |
| magi18365 | I | * | G/A | AA |
| Mo17-101121 | I | 11 | T/C | CC |
| PZA03746.1 | I | * | T/C | CC |
| PZA02737.1 | I | * | A/G | GG |
| DAS-PZ-6563 | I | 12 | A/C | CC |
| DAS-PZ-10083 | Ii | 13 | G/C | CC |
| DAS-PZ-1255 | Ii | 14 | A/G | GG |
| DAS-PZ-14161 | ii | 15 | T/C | CC |
| DAS-PZ-15602 | ii | 16 | T/C | CC |
| DAS-PZ-19469 | ii | 17 | A/G | GG |
| DAS-PZ-3132 | ii | 18 | A/G | AA |
| DAS-PZ-4347 | ii | 19 | T/C | CC |
| DAS-PZ-5837 | ii | 20 | C/G | GG |
| DAS-PZ-8111 | ii | 21 | G/T | TT |
| DAS-PZ-880 | ii | 22 | G/A | AA |
| DAS-PZ-9899 | ii | 23 | C/G | GG |
| magi99055 | ii | * | A/C | CC |
| Mo17-10988 | ii | 24 | A/C | CC |
| Mo17-11370 | ii | 25 | G/C | CC |
| Mo17-11923 | ii | 26 | T/A | AA |
| Mo17-13135 | ii | 27 | G/A | AA |
| Mo17-1316 | ii | 28 | T/A | AA |
| Mo17-13288 | ii | 29 | T/G | GG |
| PHM13823.7 | ii | * | C/T | TT |
| PHM2324.23 | ii | * | A/G | GG |
| PZA03070.9 | ii | * | T/G | GG |
| PZA00279.2 | ii | * | G/A | AA |
| DAS-PZ-2559 | iii | 30 | C/T | TT |
| DAS-PZ-3168 | iii | 31 | A/C | CC |
| DAS-PZ-3394 | iii | 32 | G/C | CC |
| magi104662 | iii | 33 | A/G | GG |
| magi52178 | iii | * | C/T | TT |
| Mo17-10465 | iii | 34 | C/T | TT |
| PHM6428.11 | iii | * | G/A | AA |
| PZA01470.1 | iii | * | G/A | AA |
| DAS-PZ-9978 | iii | 35 | C/T | TT |
| DAS-PZ-366 | iv | 36 | C/G | GG |
| KG-2566510 | iv | 37 | T/C | CC |
| KG-2624948 | iv | 38 | G/C | CC |
| Mo17-13418 | iv | 39 | A/C | CC |
| PZA00466.1 | iv | * | A/G | GG |
| PZA01272.1 | iv | * | A/G | GG |
| PZB01899.1 | iv | * | A/G | GG |
| PZB01963.1 | iv | * | A/G | GG |

* Sequence is available in public databases such as Maize GDB or Panzea.

### Example 2

Populations for validation of SNP markers associated with decreased flowering time. Three populations were developed to validate SNP markers from the four marker nucleic acid groups (i)-(iv), linked to early flowering QTL and determine earliness improvement by introgression of the QTL. An F₂ population of 1112 lines was derived from a cross between SLA21 and BU007. BU007 is obtainable from the ATCC under Accession No. PTA-9874. Two backcross (BC) populations of 171 and 156 individuals were derived from BV68×BU007 and BE1146BMR×BU007, respectively. BU007 is a DAS flint inbred line from Serbia/Mongolia and exhibits several quality agronomic characteristics such as early flowering, cold tolerance and heat tolerance. It is the early flowering donor.

Fresh leaf samples were collected and DNA extracted with a standard MagAttract 96 DNA Plant Core Kit (Qiagen, Valencia, Calif.) with a customized BioCel robot system from Agilent Technologies (Santa Clara, Calif.).

### Example 3

Phenotyping the F₂ and backcross populations. Phenotypic evaluations were conducted in Olivia, Minn. for the F₂ population and in Breckenridge, Minn. for the BC populations during the summer, 2011. Phenotypic data collected included days to anthesis (number of days for 50% tassel to shed pollen from planting date), days to silking (number of days for 1 inch of silking from the planting date). The populations segregated for flowering in a normal distribution manner in F₂ population. Transgressive segregants were not observed in this population.

### Example 4

Genotyping the F₂ and backcross populations. The KASPar™ genotyping system (KBiosciences, Hoddesdon, Hertfordshire, UK) was used to validate the SNP markers linked to early flowering QTL and determine earliness improvement by introgression of the QTL. The system is comprised of two components (1) the SNP-specific assay (a combination of three primers), and (2) the universal Reaction Mix, supplied at 2× concentration, and containing Taq polymerase enzyme, the passive reference dye, ROX, 50 mM MgCl₂, and DMSO. The three primers, allele-specific 1 (A1), allele-specific 2 (A2), and common (C1), or reverse, were designed using the assay design algorithm of the workflow manager, Kraken (KBiosciences, Hoddesdon, Hertfordshire, UK). The assay design algorithm allows for primer design on the forward reference strand or its reverse complement. PCR reaction mixes and thermocycling conditions were performed using Kbiosciences' recommended protocols.

After thermocycling was complete, allele-specific fluorescent intensities were read using a PHERAStar® Spectrofluorometer (BMG LabTech, Cary, N.C.) at room temperature and data was uploaded to the Kraken system for analysis.

The F₂ population was screened with the markers from the four marker nucleic acid groups (i)-(iv) (Table 2), to determine the number of donor loci that were present within each plant. The correlation between flowering time and the number of donor loci present was determined and a reduction of flowering time was shown when the number of donor loci in the plant increased from 0 to 4. For the SLA21×BU007 population, the elite parent SLA21 flowered after 70 days, while the donor parent BU007 flowered after 54 days. Depending on which loci and number of loci present, the F₂ progeny flowering time distribution ranged from 52 to 61 days.

The BC populations were screened with the markers from the four marker nucleic acid groups (i)-(iv) (Table 2), to determine the number of donor loci that were present within each plant. The correlation between flowering time and the number of donor loci present was determined and a reduction of flowering time was shown when the number of donor loci in the plant increases from 0 to 4. For the BV68×BU007 population, the elite parent BV68 flowered after 71 days, while the donor parent BU007 flowered after 54 days. Depending on which loci and number of loci present, the BC progeny flowering time distribution ranged from 62 to 69 days. For the BE1146BMR×BU007 population, the elite parent BE1146BMR flowered after 72 days, while the donor parent BU007 flowered after 54 days. Depending on which loci and number of loci present, the BC progeny flowering time distribution ranged from 61 to 70 days.

Analyses using the markers from the four marker nucleic acid groups (i)-(iv) and phenotypic data indicated that there was a significant reduction in flowering time when all four early flowering loci were present in both the F₂ and BC populations. Additionally, the lines without the four early flowering loci had later flowering times in the populations.

TABLE 2
Exemplary assays for detecting decreased flowering time.

| | SEQ | SEQ ID | SEQ ID | SEQ ID |
|---|---|---|---|---|
| | ID | Forward | Forward | Reverse |
| Marker | NO | primer (A1) | primer (A2) | Primer |
| DAS-PZ-10450 | 1 | 40 | 41 | 42 |
| DAS-PZ-12076 | 2 | 43 | 44 | 45 |
| DAS-PZ-16344 | 3 | 46 | 47 | 48 |
| DAS-PZ-1867 | 4 | 49 | 50 | 51 |
| DAS-PZ-2256 | 5 | 52 | 53 | 54 |
| DAS-PZ-6992 | 6 | 55 | 56 | 57 |
| DAS-PZ-8291 | 7 | 58 | 59 | 60 |
| DAS-PZ-9937 | 8 | 61 | 62 | 63 |
| DSDS-0066-1 | 9 | 64 | 65 | 66 |
| KG-2679163 | 10 | 67 | 68 | 69 |
| magi18365 | * | 70 | 71 | 72 |
| Mo17-101121 | 11 | 73 | 74 | 75 |
| PZA03746.1 | * | 76 | 77 | 78 |
| PZA02737.1 | * | 79 | 80 | 81 |
| DAS-PZ-6563 | 12 | 82 | 83 | 84 |
| DAS-PZ-10083 | 13 | 85 | 86 | 87 |
| DAS-PZ-1255 | 14 | 88 | 89 | 90 |
| DAS-PZ-14161 | 15 | 91 | 92 | 93 |
| DAS-PZ-15602 | 16 | 94 | 95 | 96 |
| DAS-PZ-19469 | 17 | 97 | 98 | 99 |
| DAS-PZ-3132 | 18 | 100 | 101 | 102 |
| DAS-PZ-4347 | 19 | 103 | 104 | 105 |
| DAS-PZ-5837 | 20 | 106 | 107 | 108 |
| DAS-PZ-8111 | 21 | 109 | 110 | 111 |
| DAS-PZ-880 | 22 | 112 | 113 | 114 |
| DAS-PZ-9899 | 23 | 115 | 116 | 117 |
| magi99055 | * | 118 | 119 | 120 |
| Mo17-10988 | 24 | 121 | 122 | 123 |
| Mo17-11370 | 25 | 124 | 125 | 126 |
| Mo17-11923 | 26 | 127 | 128 | 129 |
| Mo17-13135 | 27 | 130 | 131 | 132 |
| Mo17-1316 | 28 | 133 | 134 | 135 |
| Mo17-13288 | 29 | 136 | 137 | 138 |
| PHM13823.7 | * | 139 | 140 | 141 |
| PHM2324.23 | * | 142 | 143 | 144 |
| PZA03070.9 | * | 145 | 146 | 147 |
| PZA00279.2 | * | 148 | 149 | 150 |
| DAS-PZ-2559 | 30 | 151 | 152 | 153 |
| DAS-PZ-3168 | 31 | 154 | 155 | 156 |
| DAS-PZ-3394 | 32 | 157 | 158 | 159 |
| magi104662 | 33 | 160 | 161 | 162 |
| magi52178 | * | 163 | 164 | 165 |
| Mo17-10465 | 34 | 166 | 167 | 168 |
| PHM6428.11 | * | 169 | 170 | 171 |
| PZA01470.1 | * | 172 | 173 | 174 |
| DAS-PZ-9978 | 35 | 175 | 176 | 177 |
| DAS-PZ-366 | 36 | 178 | 179 | 180 |
| KG-2566510 | 37 | 181 | 182 | 183 |
| KG-2624948 | 38 | 184 | 185 | 186 |
| Mo17-13418 | 39 | 187 | 188 | 189 |
| PZA00466.1 | * | 190 | 191 | 192 |
| PZA01272.1 | * | 193 | 194 | 195 |
| PZB01899.1 | * | 196 | 197 | 198 |
| PZB01963.1 | * | 199 | 200 | 201 |

* Sequence is available in public databases such as Maize GDB or Panzea.

### Example 5

Introgression of decreased flowering time into a corn plant. Corn breeders can use the SNP markers provided in the present invention to introgress decreased flowering time into a corn plant. The markers provided in Table 2 can be used to monitor the introgression of early flowering time QTL into a corn plant.

The introgression of one or more early flowering loci is achieved via one or more cycles of backcrossing to a recurrent parent with one or more preferred agronomic characteristics, accompanied by selection to retain the one or more early flowering loci from the donor parent using the markers of the present invention. Introgression can be monitored by genotyping one or more plants and determining the allelic state of the one or more early flowering loci. This backcross procedure is implemented at any stage in variety development and occurs in conjunction with breeding for one or more traits of interest including transgenic and nontransgenic traits.

Alternatively, a forward breeding approach is employed wherein one or more early flowering loci can be monitored for successful introgression following a cross with a susceptible parent with subsequent generations genotyped for one or more early flowering loci and for one or more additional traits of interest, including transgenic and nontransgenic traits.

We claim:

1. A method for selecting a plant having an altered flowering characteristic, the method comprising the steps of:
a) detecting at least one marker nucleic acid; and,
b) selecting a plant comprising the marker nucleic acid, thereby selecting a plant having the altered flowering characteristic.

2. The method of claim 1, wherein the plant is a maize plant.

3. The method of claim 2, wherein the altered flowering characteristic is altered flowering time.

4. The method of claim 3, wherein the altered flowering time is decreased flowering time.

5. The method of claim 4, wherein the flowering time is decreased by at least 2 days.

6. The method of claim 5, wherein the marker nucleic acid is selected from the group consisting of DAS-PZ-10450, DAS-PZ-12076, DAS-PZ-16344, DAS-PZ-1867, DAS-PZ-2256, DAS-PZ-6992, DAS-PZ-8291, DAS-PZ-9937, DSDS-0066-1, KG-2679163, magi18365, Mo17-101121, PZA03746.1, PZA02737.1, and DAS-PZ-6563.

7. The method of claim 5, wherein the marker nucleic acid is selected from the group consisting of DAS-PZ-10083, DAS-PZ-1255, DAS-PZ-14161, DAS-PZ-15602, DAS-PZ-19469, DAS-PZ-3132, DAS-PZ-4347, DAS-PZ-5837, DAS-PZ-8111, DAS-PZ-880, DAS-PZ-9899, magi99055, Mo17-10988, Mo17-11370, Mo17-11923, Mo17-13135, Mo17-1316, Mo17-13288, PHM13823.7, PHM2324.23, PZA03070.9, and PZA00279.2

8. The method of claim 5, wherein the marker nucleic acid is selected from the group consisting of DAS-PZ-2559, DAS-PZ-3168, DAS-PZ-3394, magi104662, magi52178, Mo17-10465, PHM6428.11, PZA01470.1, DAS-PZ-3168 and DAS-PZ-9978.

9. The method of claim 5, wherein the marker nucleic acid is selected from the group consisting of DAS-PZ-366, KG-2566510, KG-2624948, Mo17-13418, PZA00466.1, PZA01272.1, PZB01899.1, and PZB01963.1.

10. The method of claim 1, wherein at least two marker nucleic acids are selected.

11. The method of claim 1, wherein at least three marker nucleic acids are selected.

12. The method of claim 1, wherein at least four marker nucleic acids are selected.

13. A maize plant obtained by the method of claim 1.

14. A method for selecting a maize plant having decreased flowering time, the method comprising:
a) detecting at least four marker nucleic acids, wherein at least one marker nucleic acid is selected from each of four marker nucleic acid groups (i)-(iv):
(i) DAS-PZ-10450, DAS-PZ-12076, DAS-PZ-16344, DAS-PZ-1867, DAS-PZ-2256, DAS-PZ-6992, DAS-PZ-8291, DAS-PZ-9937, DSDS-0066-1, KG-2679163, magi18365, Mo17-101121, PZA03746.1, PZA02737.1, and DAS-PZ-6563;
(ii) DAS-PZ-10083, DAS-PZ-1255, DAS-PZ-14161, DAS-PZ-15602, DAS-PZ-19469, DAS-PZ-3132, DAS-PZ-4347, DAS-PZ-5837, DAS-PZ-8111, DAS-PZ-880, DAS-PZ-9899, magi99055, Mo17-10988, Mo17-11370, Mo17-11923, Mo17-13135, Mo17-1316, Mo17-13288, PHM13823.7, PHM2324.23, PZA03070.9, and PZA00279.2;
(iii) DAS-PZ-2559, DAS-PZ-3168, DAS-PZ-3394, magi104662, magi52178, Mo17-10465, PHM6428.11, PZA01470.1, DAS-PZ-3168, and DAS-PZ-9978; and,
(iv) DAS-PZ-366, KG-2566510, KG-2624948, Mo17-13418, PZA00466.1, PZA01272.1, PZB01899.1, and PZB01963.1; and,
b) selecting a plant comprising the four marker nucleic acids, thereby selecting a maize plant having decreased flowering time.

15. A maize plant obtained by the method of claim 14.


SEQUENCE LISTING (201 sequences)

SEQ ID NO: 1 - DNA, 201 bp, Zea mays
ccgtcgccatcaacggcgcaggcggcgacgcgcgcgcgctgtgggcgtccgaaggcttcgcggcgtcctcgccggtcgcgccgagggtgcagcttctggtwcagctcctcaacgaggtactcgcgctctctccgtacgtcccttgcttgggcgcgggcgttgacggtctcggcggggacgcgtccgaggccaaggtggacg

SEQ ID NO: 2 - DNA, 201 bp, Zea mays
atgtgctcttcttctacgtgttttcttgcggctgggctcagcagttgcccgtgcagccgtgctcgtctagaccgtgtccatgctgctgcaagcaccaacakggttttactagtattttccacggcatctttcgcagagctcgtagctgcatgtctgagtgcctgcgatggcgattgaagaataccgtatgggcatatatat

SEQ ID NO: 3 - DNA, 201 bp, Zea mays
ttgcgctaagcccagatgtgcttcagaaggcagattgctcggtggacaaagacgagctcgaagagaagctgcagagccatttctccgtgctaagaggcatmtctgagaatgagaggttgagggcagagctgaaccacacactgtcatccataaacatgtacaaggagtacaagaagcagaaaaggaaaaacatgaaagtta

SEQ ID NO: 4 - DNA, 201 bp, Zea mays
gggcgtccagctcggctgcttccagcccgggcctgttctgtttatttttgctaggtttatgaccttgccttgcaacttgcgatacatgaccgactaggatkaggtcggttcagctccgttcaggcaaggcacggattggagctccggagcttgttagctaccttgattgtgttccaagaagatggatgagtggaagaattg

SEQ ID NO: 5 - DNA, 201 bp, Zea mays
aagggccaccaccacctcggccccgccggccagcgaagggtgctcgtcgtcgtcggcacgggtgccgacattccagtggcccccgactgcagaggaggacrccttcttcaccgagaagctgtatggccccagccgccgtcgcaggctgcccgtgttccaggacatctgccctgacgacgaaacaagccaaccccctacccc

SEQ ID NO: 6 - DNA, 201 bp, Zea mays
gtcggcgcttccggagctctgttcgggctccttggggccatgctgtcggagctcttcaccaactggaccatctatgcaaacaaggtgatgcatagcttccmtcctcggtcctaaacatcgcaatgtggatctttgggcagaaaatgtgggttgggaattaaatcgcagctcactgaagttgtaacgtccaatctgcaggct

SEQ ID NO: 7 - DNA, 201 bp, Zea mays
aggccagggggtgcctcaggttcagcgacggcggcgaccaagggaccacggcggtgtaccaggacggcagcaacgcgggcgatgtctgaatgatagatagwctctttgtgccaaaagcactatacgaatttgtattgtaggctagtgttttgtgattaggaattagtggttccggggaaaaggattcatgctccctccagt

SEQ ID NO: 8 - DNA, 201 bp, Zea mays
cggctcaactacacgggccgctcgtctcagctttcctcccgccgtagctcacctactaaacaagtgtaaaatcacgtaagatcaccgcgcgtgtcctaggmcgtacaggttggtactctaaactcgctccatgtgtgcccgccatgtgcggagacggtggtgatctaggctatagattcagccagttgcgtccggtcatct

SEQ ID NO: 9 - DNA, 201 bp, Zea mays
aaacagaaatcgtgaatgccagtagagtagcatacagatttatggcaggcctgacttccaaaaatcaagcctcacctgagtctccgggtaacaatcaaaayagaggcagccctagtttccagggagcttggttcaagaacatcgtctcagctggtgcaaagccttcatcgagtggatcgacacgcacacattttcaggacg

SEQ ID NO: 10 - DNA, 81 bp, Zea mays
tggatcgcaccatctttggatgatgtcgtcgcagaaacgayggaagggatgcatcttcttctccctcgccttgcttctctg

SEQ ID NO: 11 - DNA, 121 bp, Zea mays
cacgcggcaccggaaccgactctattactccgcgcttgtacgtacgctcatagcgacgcaygagttgcgccctgacccggttaaatgcggagcgacatacaggcacagttaaatgtggagt

SEQ ID NO: 12 - DNA, 121 bp, Zea mays
atgcgaacaatttcgagtagcgtttactgtgggaaaaataaacttgtaatccttgaatggmagaacaatcagagatactagggcggagtcggttatggaatcttttttctctatctatgtg

SEQ ID NO: 13 - DNA, 201 bp, Zea mays
caaccttgtcgataagacggagactctccgcgtgaccccgcgatccaaacgcctctaggaacaggtctgttcccatctcatccctcccagccaaacactastaagagcacgacaaagaggaagcgggaagctgagaatcagcagctcaggctcacctggtcggcgcggtccccgacgacgaggccgtgcagcgagcaccac

SEQ ID NO: 14 - DNA, 201 bp, Zea mays
ccgccagaagtacggtggagcatggccaaagtactgccaaactggcaggttagcgaactaattccctacccgaaacatcttacgcaatgtatgcttaacgrcgaacaccatgaaaagacttacagcgtgcacggaacggcgacggaagcaggccacggcaaagcgcggtccgcggcggcggtgtaatgatggtgagaaatt

SEQ ID NO: 15 - DNA, 201 bp, Zea mays
cttgaacttgaaatccgcgtgctcctgattgggtcatctcatcggcatccaagtcagaaagaacatttaacgtaaggataattgtgaagcacagatcatgyacctgagttatgagctggcgccctgattcgatcagcacttggaagccaagtgtgcccatcactgatgcgaaaactattattccctgcgataaatgtcaaa

SEQ ID NO: 16 - DNA, 201 bp, Zea mays
ggccttgcctgttttacgctcgtcgccggcaaagggccaggcggagagttggcgaatcctatccgttaagctaaacggaactacaaatcagtgctccaagyggctggactgttggtgatcgaaatcgagcaagaaaatgttgaagggaagaggagcctgaggcgacaacatacgtcggtggagtgcgccggtgctgctggt

SEQ ID NO: 17 - DNA, 201 bp, Zea mays
ccgatggcgtccaacgctgagccacccatcagctttcctggaaccggtggtcgattcctacgtcgggcgttaggaagcaaacaaggagggtcatgatcctrctacatgttcatgcctaagagttcaagggctaaggcaccaagcattgtatggtgacgaagcagcttacctttgagggcgttggcgagggacaacacgtcc

SEQ ID NO: 18 - DNA, 201 bp, Zea mays
agaacttttggagccttgcgatgattgcgttctacaggctggctttacaaaagctcagcttgatccgcctaatcaggaatctattgggttgccactgtccraagccagttgcctgactgaaaccctctgtttgctgtacctgctgtacctgtactcgcatagacatgcactgtagcgcgtcagccaattcatacgcatata

SEQ ID NO: 19 - DNA, 201 bp, Zea mays
gcgcccgcggccgtcgacacccgtcccgatacggaacggaacgggatctgcctgcccagtgcccactagcggacgattctatgtggtgttgtcacgttgtytcctaccgtcccgtatccagcgctatcgcttattttaactccaagcacccctgcctttacttgctccaggcttttgtcgagccagtgtattatattgtaa

SEQ ID NO: 20 - DNA, 201 bp, Zea mays
ctcgtcatcaagccaatcgcgagtaagaacattcttcctggatccgccgtcgtcttaccttcgactagcacctagcgtatcgggtaatcgtgagccgtggsccagtatgggattctcgacatggcgcaccgccgtggtgttctgttccgccgccggtgtttgaccgggtaggggagattgtgatcaaccgttggattcaac

SEQ ID NO: 21 - DNA, 201 bp, Zea mays
agtggaaatctaagctggttcgtccgttgttggagaatcagaaaaaaaaaaattgtcgtgcttcattcacatcacatggatacacagtttcctgttcttgkagcagaagaactggaaagatctctcgttatctctatttgctatagaaagaacatagcggcggtatctgcagacgatacaaatggaagaagagcgggcaag

SEQ ID NO: 22 - DNA, 201 bp, Zea mays
tcagcgtcgtgtttggcatcctttgttgagtgcttggacaggtatggtgcccggaagctagccatttctcgatcaatgcccgctcgtatgtctgaaaaagrcacaagcaaaaaacaactgtctcatatgacgtgttgagattgccttgtaagtacttgagtatggaaacatagaatcagatgtaggcaggcacctggccag

SEQ ID NO: 23 - DNA, 201 bp, Zea mays
aaataccgaatcgatcttgatcgctaaattgtcagcgacacgccgacgacggcatcctacaccacgaacgactagggaccaagtgaacaagacacaagagstgtacggtgtacctgccgtgccgttggccgttgcccacaggtgccaccacctgtccacctccaacgcacggctccggttgcacgggggcctctccgcttt

SEQ ID NO: 24 - DNA, 121 bp, Zea mays
cccagaaaggatggggcatgtagttgacaagtcaagaacgggtgacgggtcgctacagtgmtacgtgcattacggttaagcttatccttatccaccgattaagacatggagcaggagcggg

SEQ ID NO: 25 - DNA, 121 bp, Zea mays
gagatgggtgcgtgagtaattgactcagtcaacacggcagcgcacaaaactaaggcctccsttgaccttaaacatgcatttcgttctatctcgtctaccggaagatcgtgattgttttcgc

SEQ ID NO: 26 - DNA, 121 bp, Zea mays
cgcagccaaagctggaggtagcttatatatataatgcatgcatgtccctagcctgcatctwcagttgacatttacacatcgcggccggctggggatggatggattggcccttgagtcccgg

SEQ ID NO: 27 - DNA, 121 bp, Zea mays
gacagtgccgctgcgcctccgcattcaaaacgccgtcccgggtaggtcgtctcgattcaargcaccgctcctccgatcctacctaatcgtttggctcgtcttcttcaccttcgtcattcat

SEQ ID NO: 28 - DNA, 121 bp, Zea mays
gccgctgcgaataaactatctgcaaatcaccgtccaaagtacgaccagtcgaccacctgcwgagtggcagtactactacggtttaatctctgtacgtgtcagcctccggttggccggagtg

SEQ ID NO: 29 - DNA, 121 bp, Zea mays
cctgatcccgttcctgtttgaagcagcgatggcggcgctcaccaagtccatgtgctgggakatagtgaagaagacgagcgacacggtggacgagaccgccatggtggtgttcaagaagccg

SEQ ID NO: 30 - DNA, 201 bp, Zea mays
ggcccgtggatcgtcgccggacgccaggcatcggcaagcggcagcgcagccgtccaccctgagtccctgattttgccaaggttattagccctccctcctcygcactatctctgtaacaccaattcattctagcttacttctcatgagatgagaggggcgcatccagcaccggggcggccgctgtccgggctcgatcgagcc

SEQ ID NO: 31 - DNA, 201 bp, Zea mays
ctaggacaggtataggcagcatatatagcagcgtgcatatatccactgcacgcacgcacggatatcaaggcgatcgagaatgatgctttggttacgtactmgggaggtcccatggctcaatcttgtttaggtcggcctccgtgatggcggtgcaggtgaagcgcgtgttgagcaccttgttggtgaggtagaagctgacaa

SEQ ID NO: 32 - DNA, 201 bp, Zea mays
aaaccgtactatagataatggcccctattcaatttcatgctggagatactcatcgcagaagctggctgttggtacactagtctgcttctctttcatatggsttagatggtttttgtgtggctaaggtggaatcgatctctgtttgagatttatgtaactctgcaatgggcaatactgctttctgtaatctctatgtcggag

SEQ ID NO: 33 - DNA, 121 bp, Zea mays
ggcctaagcatgtaacgagataacaggggttccagttgggtgatgatcatttacatcatgrcgggcgaacaaaacatgagcagctacactactgctgggaagtaccgacgaacattacatt

SEQ ID NO: 34 - DNA, 121 bp, Zea mays
cggctccgcgttcgcgtggtccgaagacctcatgttcgtcgcgtccgagccgctcgacgayaacctcatcgtgctagtggaggatcggtccatgatcaaggaacccgcgctcctcggccac

SEQ ID NO: 35 - DNA, 121 bp, Zea mays
ggcaggcaggagcagagcaggaccggcgtccgggcgggacttggactgttcctgctgcaaycacacacagatcaagcactagagatgtccccaacacggggagcaagggaaacgagagaga

SEQ ID NO: 36 - DNA, 201 bp, Zea mays
ggtcacaggaacgcccggccatggcccagcgcgggtttatggcgacggcgaggaattccggcggcattggtggtagactccgagtgatacagtcacgactscactcccctggtttgtagaagaccccaggcacgacactgaagatttatgtctacggcggcgtggagttctccccttccctgcagcggcaccccctccacg

SEQ ID NO: 37 - DNA, 81 bp, Zea mays
catacaagcattcagacagttggttagtgctttcttgctcyagagtataatattttcaatctttacatgtttcggcaccgg

SEQ ID NO: 38 - DNA, 81 bp, Zea mays
tccacttcgcgatgccagccatagtgaagaactccatcacsatactctccgatgcagggctaggcatggctatgtttagct

SEQ ID NO: 39 - DNA, 121 bp, Zea mays
atcgccgccgcgtccgtcgtctaccaagccgtacgtgcactgtggaaccctcttgccaagmaatgcatacagcatacgtcgtgggcgtggtgaactgcgcgcggctgcgagtgaatgaatg

SEQ ID NO: 40 - DNA, 22 bp, Artificial Sequence
  DAS-PZ-10450_A1
gagtacctcgttgaggagctgt

SEQ ID NO: 41 - DNA, 22 bp, Artificial Sequence
  DAS-PZ-10450_A2
gagtacctcgttgaggagctga

SEQ ID NO: 42 - DNA, 19 bp, Artificial Sequence
  DAS-PZ-10450_C1
cgcgccgagggtgcagctt

SEQ ID NO: 43 - DNA, 17 bp, Artificial Sequence
  DAS-PZ-12076_A1
gctgcaagcaccaacat

SEQ ID NO: 44 - DNA, 22 bp, Artificial Sequence
  DAS-PZ-12076_A2
atgctgctgcaagcaccaacag

SEQ ID NO: 45 - DNA, 29 bp, Artificial Sequence
  DAS-PZ-12076_C1
cgaaagatgccgtggaaaatactagtaaa

SEQ ID NO: 46 - DNA, 23 bp, Artificial Sequence
  DAS-PZ-16344_A1
ccctcaacctctcattctcagat

SEQ ID NO: 47 - DNA, 23 bp, Artificial Sequence
  DAS-PZ-16344_A2
ccctcaacctctcattctcagag

SEQ ID NO: 48 - DNA, 24 bp, Artificial Sequence
  DAS-PZ-16344_C1
ccatttctccgtgctaagaggcat

SEQ ID NO: 49 - DNA, 24 bp, Artificial Sequence
  DAS-PZ-1867_A1
tgcgatacatgaccgactaggatt

SEQ ID NO: 50 - DNA, 23 bp, Artificial Sequence
  DAS-PZ-1867_A2
gcgatacatgaccgactaggatg

SEQ ID NO: 51 - DNA, 22 bp, Artificial Sequence
  DAS-PZ-1867_C1
gccttgcctgaacggagctgaa

SEQ ID NO: 52 - DNA, 20 bp, Artificial Sequence
  DAS-PZ-2256_A1
cccgactgcagaggaggaca

SEQ ID NO: 53 - DNA, 19 bp, Artificial Sequence
  DAS-PZ-2256_A2
ccgactgcagaggaggacg

SEQ ID NO: 54 - DNA, 24 bp, Artificial Sequence
  DAS-PZ-2256_C1
gccatacagcttctcggtgaagaa

SEQ ID NO: 55 - DNA, 24 bp, Artificial Sequence
  DAS-PZ-6992_A1
attgcgatgtttaggaccgaggat

SEQ ID NO: 56 - DNA, 21 bp, Artificial Sequence
  DAS-PZ-6992_A2
gcgatgtttaggaccgaggag

SEQ ID NO: 57 - DNA, 29 bp, Artificial Sequence
  DAS-PZ-6992_C1
atctatgcaaacaaggtgatgcatagctt

SEQ ID NO: 58 - DNA, 26 bp, Artificial Sequence
  DAS-PZ-8291_A1
cgtatagtgcttttggcacaaagagt

SEQ ID NO: 59 - DNA, 26 bp, Artificial Sequence
  DAS-PZ-8291_A2
cgtatagtgcttttggcacaaagaga

SEQ ID NO: 60 - DNA, 25 bp, Artificial Sequence
  DAS-PZ-8291_C1
gcgggcgatgtctgaatgatagata

SEQ ID NO: 61 - DNA, 25 bp, Artificial Sequence
  DAS-PZ-9937_A1
gagtttagagtaccaacctgtacgt

SEQ ID NO: 62 - DNA, 24 bp, Artificial Sequence
  DAS-PZ-9937_A2
agtttagagtaccaacctgtacgg

SEQ ID NO: 63 - DNA, 23 bp, Artificial Sequence
  DAS-PZ-9937_C1
gtaagatcaccgcgcgtgtccta

SEQ ID NO: 64 - DNA, 20 bp, Artificial Sequence
  DSDS-0066-1_A1
ggaaactagggctgcctctg

SEQ ID NO: 65 - DNA, 22 bp, Artificial Sequence
  DSDS-0066-1_A2
ctggaaactagggctgcctcta

SEQ ID NO: 66 - DNA, 25 bp, Artificial Sequence
  DSDS-0066-1_C1
cctgagtctccgggtaacaatcaaa

SEQ ID NO: 67 - DNA, 23 bp, Artificial Sequence
  KG-2679163_A1
gagaagaagatgcatcccttcca

SEQ ID NO: 68 - DNA, 23 bp, Artificial Sequence
  KG-2679163_A2
gagaagaagatgcatcccttccg

SEQ ID NO: 69 - DNA, 26 bp, Artificial Sequence
  KG-2679163_C1
catctttggatgatgtcgtcgcagaa

SEQ ID NO: 70 - DNA, 24 bp, Artificial Sequence
  MAGI_18365_A1
tcactagaagtggacaccacatca

SEQ ID NO: 71 - DNA, 23 bp, Artificial Sequence
  MAGI_18365_A2
cactagaagtggacaccacatcg

SEQ ID NO: 72 - DNA, 29 bp, Artificial Sequence
  MAGI_18365_C1
gtcctgtcctatgttaacctacataagaa

SEQ ID NO: 73 - DNA, 22 bp, Artificial Sequence
  Mo17-101121_A1
acgtacgctcatagcgacgcat

SEQ ID NO: 74 - DNA, 21 bp, Artificial Sequence
  Mo17-101121_A2
cgtacgctcatagcgacgcac

SEQ ID NO: 75 - DNA, 23 bp, Artificial Sequence
  Mo17-101121_C1
catttaaccgggtcagggcgcaa

SEQ ID NO: 76 - DNA, 25 bp, Artificial Sequence
  PZA03746.1_A1
cactaccatcgcaagttcctgttat

SEQ ID NO: 77 - DNA, 24 bp, Artificial Sequence
  PZA03746.1_A2
actaccatcgcaagttcctgttac

SEQ ID NO: 78 - DNA, 21 bp, Artificial Sequence
  PZA03746.1_C1
atcccggtggtggctgaggat

SEQ ID NO: 79 - DNA, 25 bp, Artificial Sequence
  PZA02737-1_A1
gattcttcgtgttgatgatcagcaa

SEQ ID NO: 80 - DNA, 21 bp, Artificial Sequence
  PZA02737-1_A2
cttcgtgttgatgatcagcag

SEQ ID NO: 81 - DNA, 27 bp, Artificial Sequence
  PZA02737-1_C1
ctgaatggcaaagcttctcttgatgct

SEQ ID NO: 82 - DNA, 25 bp, Artificial Sequence
  DAS-PZ-6563_A1
cgccctagtatctctgattgttctt

SEQ ID NO: 83 - DNA, 24 bp, Artificial Sequence
  DAS-PZ-6563_A2
gccctagtatctctgattgttctg

SEQ ID NO: 84 - DNA, 29 bp, Artificial Sequence
  DAS-PZ-6563_C1
cgagtagcgtttactgtgggaaaaataaa

SEQ ID NO: 85 - DNA, 22 bp, Artificial Sequence
  DAS-PZ-10083_A1
atccctcccagccaaacactac

SEQ ID NO: 86 - DNA, 22 bp, Artificial Sequence
  DAS-PZ-10083_A2
atccctcccagccaaacactag

SEQ ID NO: 87 - DNA, 24 bp, Artificial Sequence
  DAS-PZ-10083_C1
cgcttcctctttgtcgtgctctta

SEQ ID NO: 88 - DNA, 23 bp, Artificial Sequence
  DAS-PZ-1255_A1
gtaagtcttttcatggtgttcgt

SEQ ID NO: 89 - DNA, 25 bp, Artificial Sequence
  DAS-PZ-1255_A2
ctgtaagtcttttcatggtgttcgc

SEQ ID NO: 90 - DNA, 29 bp, Artificial Sequence
  DAS-PZ-1255_C1
ccgaaacatcttacgcaatgtatgcttaa

SEQ ID NO: 91 - DNA, 22 bp, Artificial Sequence
  DAS-PZ-14161_A1
gcgccagctcataactcaggta

SEQ ID NO: 92 - DNA, 21 bp, Artificial Sequence
  DAS-PZ-14161_A2
cgccagctcataactcaggtg

SEQ ID NO: 93 - DNA, 29 bp, Artificial Sequence
  DAS-PZ-14161_C1
cgtaaggataattgtgaagcacagatcat

SEQ ID NO: 94 - DNA, 21 bp, Artificial Sequence
  DAS-PZ-15602_A1
cgatcaccaacagtccagcca

SEQ ID NO: 95 - DNA, 21 bp, Artificial Sequence
  DAS-PZ-15602_A2
cgatcaccaacagtccagccg

SEQ ID NO: 96 - DNA, 28 bp, Artificial Sequence
  DAS-PZ-15602_C1
taaacggaactacaaatcagtgctccaa

SEQ ID NO: 97 - DNA, 24 bp, Artificial Sequence
  DAS-PZ-19469_A1
caaacaaggagggtcatgatccta

SEQ ID NO: 98 - DNA, 23 bp, Artificial Sequence
  DAS-PZ-19469_A2
aaacaaggagggtcatgatcctg

SEQ ID NO: 99 - DNA, 26 bp, Artificial Sequence
  DAS-PZ-19469_C1
gcccttgaactcttaggcatgaacat

SEQ ID NO: 100 - DNA, 24 bp, Artificial Sequence
  DAS-PZ-3132_A1
aatctattgggttgccactgtcca

SEQ ID NO: 101 - DNA, 21 bp, Artificial Sequence
  DAS-PZ-3132_A2
ctattgggttgccactgtccg

SEQ ID NO: 102 - DNA, 25 bp, Artificial Sequence
  DAS-PZ-3132_C1
caaacagagggtttcagtcaggcaa

SEQ ID NO: 103 - DNA, 26 bp, Artificial Sequence
  DAS-PZ-4347_A1
attctatgtggtgttgtcacgttgtt

SEQ ID NO: 104 - DNA, 23 bp, Artificial Sequence
  DAS-PZ-4347_A2
ctatgtggtgttgtcacgttgtc

SEQ ID NO: 105 - DNA, 22 bp, Artificial Sequence
  DAS-PZ-4347_C1
tagcgctggatacgggacggta

SEQ ID NO: 106 - DNA, 23 bp, Artificial Sequence
  DAS-PZ-5837_A1
catgtcgagaatcccatactggg

SEQ ID NO: 107 - DNA, 23 bp, Artificial Sequence
  DAS-PZ-5837_A2
catgtcgagaatcccatactggc

SEQ ID NO: 108 - DNA, 25 bp, Artificial Sequence
  DAS-PZ-5837_C1
actagcacctagcgtatcgggtaat

SEQ ID NO: 109 - DNA, 26 bp, Artificial Sequence
  DAS-PZ-8111_A1
gagagatctttccagttcttctgcta

SEQ ID NO: 110 - DNA, 25 bp, Artificial Sequence
  DAS-PZ-8111_A2
agagatctttccagttcttctgctc

SEQ ID NO: 111 - DNA, 29 bp, Artificial Sequence
  DAS-PZ-8111_C1
atcacatggatacacagtttcctgttctt

SEQ ID NO: 112 - DNA, 23 bp, Artificial Sequence
  DAS-PZ-880_A1
cccgctcgtatgtctgaaaaagg

SEQ ID NO: 113 - DNA, 23 bp, Artificial Sequence
  DAS-PZ-880_A2
cccgctcgtatgtctgaaaaaga

SEQ ID NO: 114 - DNA, 29 bp, Artificial Sequence
  DAS-PZ-880_C1
tctcaacacgtcatatgagacagttgttt

SEQ ID NO: 115 - DNA, 24 bp, Artificial Sequence
  DAS-PZ-9899_A1
accaagtgaacaagacacaagagc

SEQ ID NO: 116 - DNA, 24 bp, Artificial Sequence
  DAS-PZ-9899_A2
accaagtgaacaagacacaagagg

SEQ ID NO: 117 - DNA, 21 bp, Artificial Sequence
  DAS-PZ-9899_C1
cggcacggcaggtacaccgta

SEQ ID NO: 118 - DNA, 27 bp, Artificial Sequence
  MAGI_99055_A1
catgcagtgatggtttgtagtagtata

SEQ ID NO: 119 - DNA, 26 bp, Artificial Sequence
  MAGI_99055_A2
atgcagtgatggtttgtagtagtatc

SEQ ID NO: 120 - DNA, 29 bp, Artificial Sequence
  MAGI_99055_C1
caaggcgtgatattgacacatgcaatata

SEQ ID NO: 121 - DNA, 26 bp, Artificial Sequence
  Mo17-10988_A1
gataagcttaaccgtaatgcacgtat

SEQ ID NO: 122 - DNA, 26 bp, Artificial Sequence
  Mo17-10988_A2
gataagcttaaccgtaatgcacgtag

SEQ ID NO: 123 - DNA, 22 bp, Artificial Sequence
  Mo17-10988_C1
aagaacgggtgacgggtcgcta

SEQ ID NO: 124 - DNA, 27 bp, Artificial Sequence
  Mo17-11370_A1
agaacgaaatgcatgtttaaggtcaag

SEQ ID NO: 125 - DNA, 27 bp, Artificial Sequence
  Mo17-11370_A2
agaacgaaatgcatgtttaaggtcaac

SEQ ID NO: 126 - DNA, 25 bp, Artificial Sequence
  Mo17-11370_C1
tcaacacggcagcgcacaaaactaa

SEQ ID NO: 127 - DNA, 22 bp, Artificial Sequence
  Mo17-11923_A1
gcatgtccctagcctgcatcta

SEQ ID NO: 128 - DNA, 22 bp, Artificial Sequence
  Mo17-11923_A2
gcatgtccctagcctgcatctt

SEQ ID NO: 129 - DNA, 22 bp, Artificial Sequence
  Mo17-11923_C1
cagccggccgcgatgtgtaaat

SEQ ID NO: 130 - DNA, 23 bp, Artificial Sequence
  Mo17-13135_A1
cgggtaggtcgtctcgattcaaa

SEQ ID NO: 131 - DNA, 22 bp, Artificial Sequence
  Mo17-13135_A2
gggtaggtcgtctcgattcaag

SEQ ID NO: 132 - DNA, 25 bp, Artificial Sequence
  Mo17-13135_C1
gacgagccaaacgattaggtaggat

SEQ ID NO: 133 - DNA, 24 bp, Artificial Sequence
  Mo17-1316_A1
aaaccgtagtagtactgccactct

SEQ ID NO: 134 - DNA, 24 bp, Artificial Sequence
  Mo17-1316_A2
aaaccgtagtagtactgccactca

SEQ ID NO: 135 - DNA, 29 bp, Artificial Sequence
  Mo17-1316_C1
aaactatctgcaaatcaccgtccaaagta

SEQ ID NO: 136 - DNA, 22 bp, Artificial Sequence
  Mo17-13288_A1
caccaagtccatgtgctgggat

SEQ ID NO: 137 - DNA, 21 bp, Artificial Sequence
  Mo17-13288_A2
accaagtccatgtgctgggag

SEQ ID NO: 138 - DNA, 24 bp, Artificial Sequence
  Mo17-13288_C1
cgtgtcgctcgtcttcttcactat

SEQ ID NO: 139 - DNA, 22 bp, Artificial Sequence
  PHM13823.7_A1
aagctattggctgagccgtgca

SEQ ID NO: 140 - DNA, 20 bp, Artificial Sequence
  PHM13823.7_A2
gctattggctgagccgtgcg

SEQ ID NO: 141 - DNA, 25 bp, Artificial Sequence
  PHM13823.7_C1
gaggacgacaagaagatgggatctt

SEQ ID NO: 142 - DNA, 30 bp, Artificial Sequence
  PHM2324.23_A1
attttgtgtttgttatcttggtctgtatga

SEQ ID NO: 143 - DNA, 25 bp, Artificial Sequence
  PHM2324.23_A2
gtgtttgttatcttggtctgtatgg

SEQ ID NO: 144 - DNA, 29 bp, Artificial Sequence
  PHM2324.23_C1
caatacatgttcctgcaaaacaactccaa

SEQ ID NO: 145 - DNA, 22 bp, Artificial Sequence
  PZA03070.9_A1
ccaagcaaaatggtgtccctcc

SEQ ID NO: 146 - DNA, 23 bp, Artificial Sequence
  PZA03070.9_A2
accaagcaaaatggtgtccctca

SEQ ID NO: 147 - DNA, 25 bp, Artificial Sequence
  PZA03070.9_C1
gcatcgggatttcatccgtcatcaa

SEQ ID NO: 148 - DNA, 21 bp, Artificial Sequence
  PZA00279.2_A1
cggggtttctgagccatctct

SEQ ID NO: 149 - DNA, 21 bp, Artificial Sequence
  PZA00279.2_A2
cggggtttctgagccatctcc

SEQ ID NO: 150 - DNA, 24 bp, Artificial Sequence
  PZA00279.2_C1
gcagagagatgcaagtgtgagcaa

SEQ ID NO: 151 - DNA, 22 bp, Artificial Sequence
  DAS-PZ-2559_A1
ggttattagccctccctcctct

SEQ ID NO: 152 - DNA, 21 bp, Artificial Sequence
  DAS-PZ-2559_A2
gttattagccctccctcctcc

SEQ ID NO: 153 - DNA, 30 bp, Artificial Sequence
  DAS-PZ-2559_C1
aagctagaatgaattggtgttacagagata

SEQ ID NO: 154 - DNA, 26 bp, Artificial Sequence
  DAS-PZ-3168_A1
gagaatgatgctttggttacgtacta

SEQ ID NO: 155 - DNA, 26 bp, Artificial Sequence
  DAS-PZ-3168_A2
gagaatgatgctttggttacgtactc

SEQ ID NO: 156 - DNA, 25 bp, Artificial Sequence
  DAS-PZ-3168_C1
gccgacctaaacaagattgagccat

SEQ ID NO: 157 - DNA, 23 bp, Artificial Sequence
  DAS-PZ-3394_A1
agtctgcttctctttcatatggc

SEQ ID NO: 158 - DNA, 25 bp, Artificial Sequence
  DAS-PZ-3394_A2
ctagtctgcttctctttcatatggg

SEQ ID NO: 159 - DNA, 25 bp, Artificial Sequence
  DAS-PZ-3394_C1
gatcgattccaccttagccacacaa

SEQ ID NO: 160 - DNA, 21 bp, Artificial Sequence
  MAGI_104662_A1
gctcatgttttgttcgcccgt

SEQ ID NO: 161 - DNA, 21 bp, Artificial Sequence
  MAGI_104662_A2
gctcatgttttgttcgcccgc

SEQ ID NO: 162 - DNA, 29 bp, Artificial Sequence
  MAGI_104662_C1
tccagttgggtgatgatcatttacatcat

SEQ ID NO: 163 - DNA, 27 bp, Artificial Sequence
  MAGI_52178_A1
gttatgttaagtatccaatgtctgtcg

SEQ ID NO: 164 - DNA, 28 bp, Artificial Sequence
  MAGI_52178_A2
ggttatgttaagtatccaatgtctgtca

SEQ ID NO: 165 - DNA, 28 bp, Artificial Sequence
  MAGI_52178_C1
aaagctttcaagccatgtcagatgtcaa

SEQ ID NO: 166 - DNA, 23 bp, Artificial Sequence
  Mo17-10465_A1
cctccactagcacgatgaggtta

SEQ ID NO: 167 - DNA, 22 bp, Artificial Sequence
  Mo17-10465_A2
ctccactagcacgatgaggttg

SEQ ID NO: 168 - DNA, 23 bp, Artificial Sequence
  Mo17-10465_C1
gcgtggtccgaagacctcatgtt

SEQ ID NO: 169 - DNA, 22 bp, Artificial Sequence
  PHM6428.11_A1
agttgtgctccaacaagcggct

SEQ ID NO: 170 - DNA, 21 bp, Artificial Sequence
  PHM6428.11_A2
gttgtgctccaacaagcggcc

SEQ ID NO: 171 - DNA, 25 bp, Artificial Sequence
  PHM6428.11_C1
caactcgtgcagctgtcagcaatat

SEQ ID NO: 172 - DNA, 22 bp, Artificial Sequence
  PZA01470-1_A1
ccagctgcgtgtgcatgagtta

SEQ ID NO: 173 - DNA, 21 bp, Artificial Sequence
  PZA01470-1_A2
cagctgcgtgtgcatgagttg

SEQ ID NO: 174 - DNA, 29 bp, Artificial Sequence
  PZA01470-1_C1
caacatatatctcttgcgcctctcaaaat

SEQ ID NO: 175 - DNA, 23 bp, Artificial Sequence
  DAS-PZ-9978_A1
acttggactgttcctgctgcaat

SEQ ID NO: 176 - DNA, 22 bp, Artificial Sequence
  DAS-PZ-9978_A2
cttggactgttcctgctgcaac

SEQ ID NO: 177 - DNA, 25 bp, Artificial Sequence
  DAS-PZ-9978_C1
gttggggacatctctagtgcttgat

SEQ ID NO: 178 - DNA, 22 bp, Artificial Sequence
  DAS-PZ-366_A1
ccgagtgatacagtcacgactc

SEQ ID NO: 179 - DNA, 22 bp, Artificial Sequence
  DAS-PZ-366_A2
ccgagtgatacagtcacgactg

SEQ ID NO: 180 - DNA, 23 bp, Artificial Sequence
  DAS-PZ-366_C1
gtcgtgcctggggtcttctacaa

SEQ ID NO: 181 - DNA, 25 bp, Artificial Sequence
  KG-2566510_A1
cagttggttagtgctttcttgctct

SEQ ID NO: 182 - DNA, 24 bp, Artificial Sequence
  KG-2566510_A2
agttggttagtgctttcttgctcc

SEQ ID NO: 183 - DNA, 29 bp, Artificial Sequence
  KG-2566510_C1
cggtgccgaaacatgtaaagattgaaaat

SEQ ID NO: 184 - DNA, 21 bp, Artificial Sequence
  KG-2624948_A1
agccctgcatcggagagtatg

SEQ ID NO: 185 - DNA, 21 bp, Artificial Sequence
  KG-2624948_A2
agccctgcatcggagagtatc

SEQ ID NO: 186 - DNA, 25 bp, Artificial Sequence
  KG-2624948_C1
tgccagccatagtgaagaactccat

SEQ ID NO: 187 - DNA, 20 bp, Artificial Sequence
  Mo17-13418_A1
gtggaaccctcttgccaaga

SEQ ID NO: 188 - DNA, 20 bp, Artificial Sequence
  Mo17-13418_A2
gtggaaccctcttgccaagc

SEQ ID NO: 189 - DNA, 24 bp, Artificial Sequence
  Mo17-13418_C1
cccacgacgtatgctgtatgcatt

SEQ ID NO: 190 - DNA, 26 bp, Artificial Sequence
  PZA00466-1_A1
caggcatccatcatctgtatctcaat

SEQ ID NO: 191 - DNA, 25 bp, Artificial Sequence
  PZA00466-1_A2
aggcatccatcatctgtatctcaac

SEQ ID NO: 192 - DNA, 22 bp, Artificial Sequence
  PZA00466-1_C1
acgggcacgagcttgatgcgaa

SEQ ID NO: 193 - DNA, 25 bp, Artificial Sequence
  PZA01272-1_A1
ggggctttggctgacattatatcta

SEQ ID NO: 194 - DNA, 24 bp, Artificial Sequence
  PZA01272-1_A2
gggctttggctgacattatatctg

SEQ ID NO: 195 - DNA, 26 bp, Artificial Sequence
  PZA01272-1_C1
ggccaacgtggtaaactgcaattcaa

SEQ ID NO: 196 - DNA, 22 bp, Artificial Sequence
  PZB01899.1_A1
gtgtgggaacttgtactgcgca

SEQ ID NO: 197 - DNA, 20 bp, Artificial Sequence
  PZB01899.1_A2
gtgggaacttgtactgcgcg

SEQ ID NO: 198 - DNA, 24 bp, Artificial Sequence
  PZB01899.1_C1
cttgcagtcgtgcttgtcggagta

SEQ ID NO: 199 - DNA, 23 bp, Artificial Sequence
  PZB01963.1_A1
gaacctttgtcgagctaccaact

SEQ ID NO: 200 - DNA, 23 bp, Artificial Sequence
  PZB01963.1_A2
gaacctttgtcgagctaccaacc

SEQ ID NO: 201 - DNA, 29 bp, Artificial Sequence
  PZB01963.1_C1
gaaatggaaaaggagccgcataaactatt